(19) United States
(12) Patent Application Publication
(10) Pub. No.: US 2026/0341524 A1
(43) Pub. Date: Oct. 29, 2026

(54) METHODS AND SYSTEMS FOR PREDICTIVE HEALTH ANALYSIS

(71) Applicant: Aeternum, LLC, Dulles, VA (US)

(72) Inventors: Paul Carter, Paeonian Springs, VA (US); Rosemary Carter, Paeonian Springs, VA (US); Sebastian Carter, Paeonian Springs, VA (US); Rikard Lundqvist, Ashburn, VA (US)

(21) Appl. No.: 19/653,638

(22) Filed: Apr. 21, 2026

Related U.S. Application Data
(63) Continuation-in-part of application No. 18/200,961, filed on May 23, 2023, now Pat. No. 12,638,431.

(51) Int. Cl. G16H 50/30; G06N 20/00; G16H 50/80

(52) U.S. Cl. CPC G16H 50/30; G16H 50/80; G06N 20/00

(57) ABSTRACT

Aspects of the disclosure relate to a predictive analysis platform for predicting health risks. The platform may receive sets of measurement information (e.g., from sensors or wearable devices) corresponding to health risks at a geographic location. The platform may use a predictive analysis model (e.g., a machine learning model) to generate risk scores based on the sets of measurement information. The risk scores may indicate a likelihood of the presence of a health risk at the geographic location. The platform may generate a mapping comprising the risk scores and geographic location and generate, using the predictive analysis model, a predictive analysis. The platform may output the predictive analysis to one or more computing devices.

### CROSS-REFERENCE TO RELATED APPLICATIONS

This application is a continuation-in-part of U.S. application Ser. No. 18/200,961, titled “SYSTEM FOR AIR COMPOSITION ESTIMATION”, filed May 23, 2023, which is hereby incorporated by reference in its entirety for all purposes.

### BACKGROUND

Aspects of the disclosure relate to performing air composition estimation and predictive health analysis using a device or system of devices. In some instances, sensors may be used to gather information corresponding to an environment (e.g., a geographical region, a building interior, and/or other environments). In some examples, based on the gathered information, individuals or organizations might desire to know specifically what particles are included in the air composition of the environment. Accordingly, it may be important to improve the efficiency of air composition estimation. Additionally, over time the air composition may change. Accordingly, it may also be important to identify methods for incorporating such changes into the air composition estimation process.

Further, individuals are exposed to harmful particulate matter, chemicals, gases, and metals on a daily basis, in the environments described above. As some examples of the potential harm, particulate matter, dust, benzene, beryllium, cadmium, chromium, ozone, and other substances may cause damage to the respiratory system. Multiple cardiovascular effects have also been attributed to air pollutants, including particulate matter and nitrogen dioxide. Additionally, per- and polyfluoroalkyl substances (PFAS), which may be present in consumer products such as kitchenware, clothing, food packaging, personal care products, cleaning products, paints, and electronics, may accumulate in individuals and have been linked to health issues. Other environmental toxins, such as arsenic, asbestos, radon, vinyl chloride, and formaldehyde, have also been linked to various health conditions. While laboratory tests exist that may measure some environmental toxins, such tests may be costly, cumbersome to administer, require multiple instruments to collect and analyze each substance, and may not provide real time or continuous testing. Accordingly, it would be beneficial to provide methods and systems for predictive health analysis using information gathered by wearable devices that can give accurate and real time information on the health or environmental conditions around the individual, in addition to the measurement information from the static sensors described above. Additionally, while some models have been developed to analyze medical records and lifestyle information to provide risk estimates for diseases, there are currently no models that can ingest all of the data described herein for an individual, population, and/or region based on environmental exposure, provide a risk score, and do so in real time. Accordingly, it may be important to develop systems and methods for gathering measurement information from a variety of sources described herein, in real time, and using such information to output an accurate and efficient predictive analysis of health risks, based on a comprehensive set of measurement information, for individuals, populations, or geographic regions.

### SUMMARY

Aspects of the disclosure provide effective, efficient, scalable, and convenient technical solutions that address and overcome the technical problems associated with predictive analysis. In accordance with one or more embodiments of the disclosure, a computing platform with at least one processor, a communication interface, and memory storing computer-readable instructions may receive a set of environment information corresponding to an environment from one or more environment information sources. The set of environment information may include one or more measured variables corresponding to one or more attributes of a known particle, and/or one or more additional environment variables. The computing platform may generate an estimated air composition using an environment analysis model. The computing platform may generate a cumulative particle score based on the estimated air composition. The computing platform may further determine whether or not the cumulative particle score satisfies a criteria. The criteria may indicate whether the environment is a compliant environment or an outlier environment. The computing platform may send one or more commands directing a user device to display a user interface, based on the determination. The user interface may include a notification indicating a result of the determination.

In accordance with one or more examples of the disclosure, a computing platform with at least one processor, a communication interface, and memory storing computer-readable instructions may receive, via the communication interface from one or more measurement information sources, a set of measurement information corresponding to health parameters at a geographic location. The health parameters may be or include information corresponding to a health risk (e.g., an environmental/natural disaster, a disease outbreak or viral outbreak, a hazardous temperature, a toxic spill, or the like) and information that is neutral to, or counteracts, the health risk (e.g., wind speed or direction for dispersing a chemical or toxin in the air, precipitation information for putting out a fire, or the like). In this way, the set of measurement information may include both environmental information as described herein as well as additional health information such as biometrics from an individual. The computing platform may generate, using a predictive analysis model and the set of measurement information, one or more risk scores indicating a likelihood of the presence of a health risk at the geographic location. The computing platform may generate a mapping comprising the one or more risk scores and the geographic location. The computing platform may generate, using the predictive analysis model and based on the mapping and the one or more risk scores, a predictive analysis. The predictive analysis may comprise an explanation of the one or more risk scores and an indication of the health risk at the geographic location. The computing platform may output, via the communication interface to a computing device, the predictive analysis.

As a brief description of the concepts described further herein, to solve the limitations and technical problems associated with environment analysis and/or predictive health analysis, some aspects of the disclosure relate to a system for air composition estimation. Individuals or organizations might desire to know which particles are included in the air composition of a particular environment. In order to identify the air composition, one or more environment information sources (e.g., sensors, satellites, detectors, and/or other information sources) may be implemented in order to gather environment information of the particular environment. In some instances, one or more types of particles included in the air composition (e.g., one or more specific gas molecules, one or more specific particulates, one or more specific types of dust, one or more specific biological contaminants, and/or one or more other types of particles) may be difficult to identify. Accordingly, a system may include one or more particle sensors configured to identify the one or more environment variables corresponding to one or more attributes of a known particle, in order to identify the one or more types of particles.

In these instances, the system may further include a computing platform configured to identify, using an environment analysis model (i.e., a machine learning model) and based on the variables corresponding to one or more attributes of a known particle, the types of particles detected by the particle sensors and generate an estimated air composition. The environment analysis model may be configured to take the variables as inputs. Additionally, the environment analysis model may be configured to take additional environment information (e.g., wind speed, wind direction, vibration information, and/or other information) as inputs. In some examples, the computing platform may receive the additional environment information from sources of environment information different from the particle sensors.

More particularly to solve the limitations and technical problems associated with predictive health analysis, some aspects of the disclosure relate to methods and system for predictive health analysis that involve gathering sets of measurement information from a variety of different sources, including individual wearable devices, and using the comprehensive nature of the sets of measurement information to more accurately and efficiently identify or predict health risks before they occur. The sets of measurement information may be comprehensive because they cover at least one type of information from each of the following three categories of information: information gathered by a wearable device (e.g., biometric information of the wearer of the wearable device, particulate matter measurements, chemical measurements, heavy metal measurements, gas measurements, environmental toxin measurements, per- and polyfluoroalkyl substance (PFAS) measurements, audio measurements, wind speed, wind direction, particle concentration, gas concentration, temperature, relative humidity, air pressure, dew point, vibration frequency, noise level, ventilation status, altitude, topographical information, occupancy, traffic counts, mold growth information, mildew growth information, population information, luminosity information, precipitation information, bacteria detection information, or virus detection information), information gathered by one or more static environmental sensors (e.g., particulate matter measurements, chemical measurements, heavy metal measurements, gas measurements, environmental toxin measurements, PFAS measurements, audio measurements, wind speed, wind direction, particle concentration, gas concentration, temperature, relative humidity, air pressure, dew point, vibration frequency, noise level, ventilation status, altitude, topographical information, occupancy, traffic counts, mold growth information, mildew growth information, population information, luminosity information, precipitation information, or virus detection information), and information corresponding to a healthcare service provider (e.g., therapeutic information for treating diseases, pathology information, community disease information, infection rates for a disease or virus, or infection vectors for a disease or virus). In these instances, a predictive analysis may provide an accurate and real time indication of health risks with information that is useful for an individual, population, or region to avoid or treat the health risk. This information may also be valuable to pharmaceutical corporations, insurers, medical professionals, and other individuals or entities associated with responding to health risks. The predictive analysis may be generated using a predictive analysis model (i.e., a machine learning model) based on input of the sets of measurement information. The predictive analysis may be output to the individuals and entities described above via a computing device and/or a wearable device.

These features, along with many others, are discussed in greater detail below.

### BRIEF DESCRIPTION OF THE DRAWINGS

The present disclosure is illustrated by way of example and not limited in the accompanying figures in which like reference numerals indicate similar elements and in which:

FIG. 1 depicts an illustrative environment information network for gathering information corresponding to an environment and sending the information to a computing platform for air composition estimation in accordance with one or more example embodiments;

FIG. 2A depicts an illustrative event flow for air composition estimation using an environment analysis model in accordance with one or more example embodiments;

FIG. 2B depicts an illustrative event flow for training an environment analysis model in accordance with one or more example embodiments;

FIG. 3 depicts an illustrative system for gathering variable information using a sensor in accordance with one or more example embodiments;

FIG. 4 depicts an illustrative system for gathering particle composition information using a sensor in accordance with one or more example embodiments;

FIG. 5 depicts illustrative graphical user interfaces depicting particle score bands generated in accordance with one or more example embodiments;

FIGS. 6A-6B depict illustrative computing systems in accordance with one or more example embodiments;

FIG. 7 depicts an illustrative method for air composition estimation in accordance with one or more example embodiments;

FIGS. 8A-8C depict illustrative graphical user interfaces depicting air composition estimation in accordance with one or more example embodiments;

FIG. 9 depicts an illustrative environment information network for gathering measurement information and sending the information to a computing platform for predictive analysis in accordance with one or more example embodiments;

FIG. 10 depicts an illustrative system for predictive analysis using measurement information sources and measurement information in accordance with one or more example embodiments;

FIGS. 11A-11B depict illustrative computing systems for predictive analysis in accordance with one or more example embodiments; and

FIG. 12 depicts an illustrative method for predictive analysis in accordance with one or more example embodiments.

### DETAILED DESCRIPTION

In the following description of various illustrative embodiments, reference is made to the accompanying drawings, which form a part hereof, and in which is shown, by way of illustration, various embodiments in which aspects of the disclosure may be practiced. In some instances, other embodiments may be utilized, and structural and functional modifications may be made, without departing from the scope of the present disclosure.

It is noted that various connections between elements are discussed in the following description. It is noted that these connections are general and, unless specified otherwise, may be direct or indirect, wired or wireless, and that the specification is not intended to be limiting in this respect.

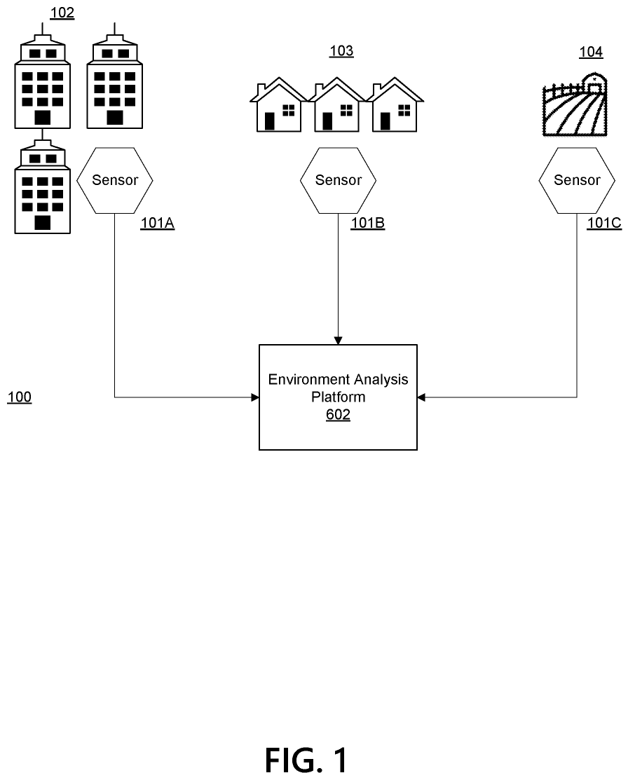

FIG. 1 depicts an illustrative environment information network for gathering information corresponding to an environment and sending the information to a computing platform (e.g., an environment analysis platform, such as environment analysis platform 602) for air composition estimation. Referring to FIG. 1, environment information network 100 may include one or more environment information sources. For example, environment information network 100 may include one or more particle sensors configured to gather variables. The one or more particle sensors may be deployed in a geographical region. For example, one or more particle sensors, such as first particle sensor 101A, may be deployed in urban region 102 (e.g., a city, town, industrial district, and/or other urban regions). In some examples, the one or more particle sensors may be deployed to one or more different geographical regions. For example, a first particle sensor 101A might be deployed in urban region 102, while one or more particle sensors such as second particle sensor 101B may be deployed in residential region 103 (e.g., a neighborhood, residential district, apartment complex, and/or other residential regions). Additionally or alternatively, in some instances, one or more particle sensors such as third particle sensor 101C may be deployed in rural region 104 (e.g., a farm, preservation, forest, road, and/or other rural regions).

In some examples, the one or more environment information sources may be communicatively coupled (e.g., via a wireless data connection) with a computing platform (e.g., for estimating air composition). For example, first particle sensor 101A, second particle sensor 101B, third particle sensor 101C, and/or other environment information sources may be communicatively coupled to environment analysis platform 602. Each of 101A, 101B, and 101C may each represent multiple particle sensors. In some examples, the computing platform may receive environment information (e.g. periodically, continuously, near continuously) from each of the environment information sources. For example, environment analysis platform 602 may continuously receive environment information from each of first particle sensor 101A, second particle sensor 101B, and third particle sensor 101C. In other instances, the computing platform may only receive environment information from an environment information source at times the environment information source is considered “active” (e.g., when the environment information source is powered on, when the environment information source is actively gathering environment information, when the environment information source is actively transmitting environment information, and/or other times the environment information source may be considered “active”).

The environment information gathered by the one or more environment information sources and received by the computing platform may include one or more measured variables corresponding to one or more attributes of a known particle, and/or one or more additional environment variables. The one or more additional environment variables may include one or more of: wind speed, wind direction, particle concentrations, gas concentrations, temperature, relative humidity, air pressure, dew point, vibration frequency, noise level, ventilation status, altitude, topographical information, occupancy, traffic count, mold growth information, mildew growth information, population information, luminosity information, precipitation information, or virus detection information. In some instances, a single environment information source (e.g., first particle sensor 101A, and/or other environment information sources) may gather a set of environment information. For example, first particle sensor 101A may gather both a measured particle count and a measured particle size.

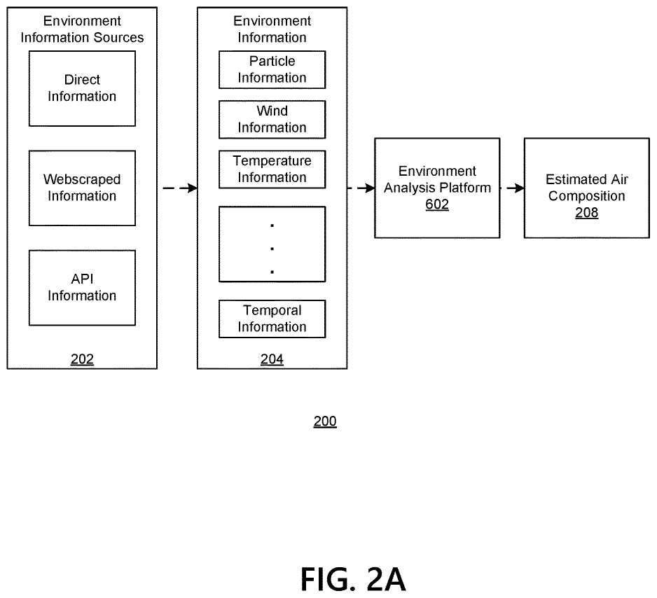

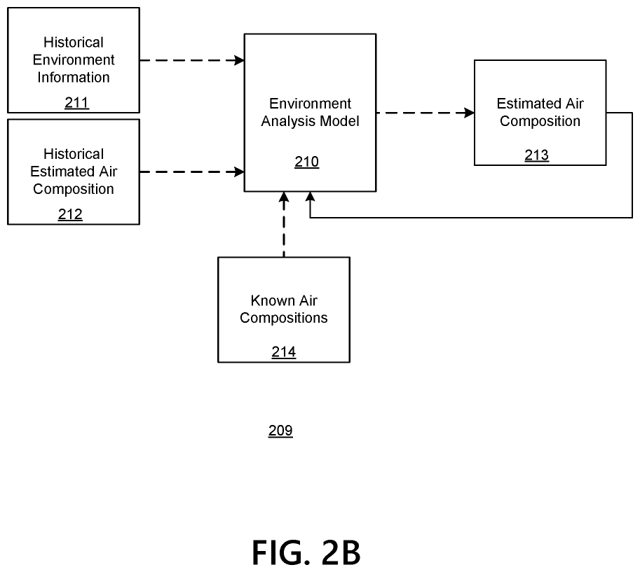

FIG. 2A depicts an illustrative event flow for air composition estimation using an environment analysis model. Referring to FIG. 2A, one or more environment information sources 202 may be configured to gather environment information 204 corresponding to a particular environment and send the environment information to the environment analysis platform 602. The one or more environment information sources 202 may be or include one or more of: a particle sensor, a network of particle sensors located at different geographical locations, a repository of information gathered by multiple unassociated sensors, satellites gathering environmental information, a repository of automotive traffic information (e.g., a repository of information gathered by one or more of cameras, radar sensors, lidar sensors, road cables, and/or other sources of traffic information), one or more particle sensors gathering environment information 204 of the interior of a building, one or more devices gathering environment information 204 from a moving vehicle, one or more anemometers, or a database corresponding to an environment analysis organization. In some examples, the one or more environment information sources may be configured to send the environment information 204 directly to the environment analysis platform 602. Additionally or alternatively, in some instances the one or more environment information sources may be configured to indirectly send the environment information 204 to the environment analysis platform 602. For example, the one or more environment information sources may send the environment information 204 to a repository (e.g., a web-based server, database, cloud storage system, and/or other repositories). In these examples, the environment analysis platform 602 may retrieve the environment information from the repository (e.g., via webscraping methods, application programming interface (API) calls, information transfer requests, and/or other methods).

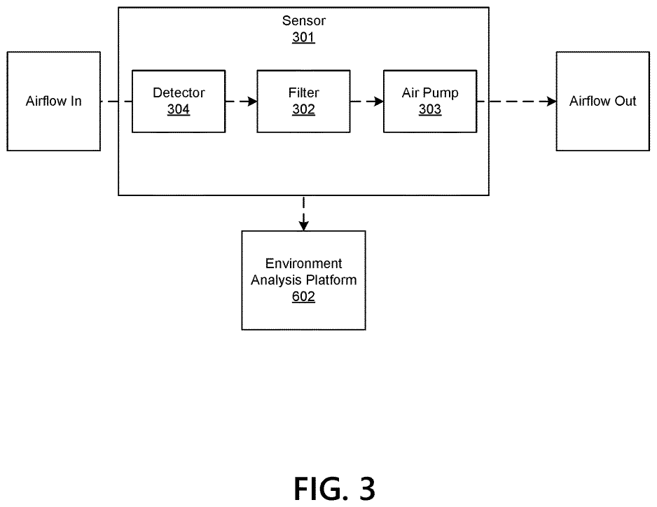

The environment information sources 202 may gather the environment information 204 corresponding to the particular environment. The environment information sources 202 may gather the environment information 204 by using cameras, lasers, filtration devices, anemometers, and/or other methods. In some instances, the environment information sources 202 may gather the environment information 204 by collecting and analyzing particles (which may, e.g., include one or more of: gas molecules, particulates, dusts, biological contaminants, aerosols, and/or other types of particles) in the air of the environment (e.g., by equipping a particle sensor with a filter to capture particles, such as in system 300, as illustrated in FIG. 3, and/or by other methods). The environment information 204 gathered by the one or more environment information sources 202 and received by the environment analysis platform 602 may include one or more measured variables corresponding to one or more attributes of a known particle, and/or one or more additional environment variables. The one or more additional environment variables may include one or more of: wind speed, wind direction, particle concentrations, gas concentrations, temperature information, relative humidity information, air pressure information, dew point information, vibration information, noise information, environment ventilation status, altitude information, topographical information, occupancy information, traffic counts, mold growth information, mildew growth information, population information, luminosity information, precipitation information, and/or virus detection information.

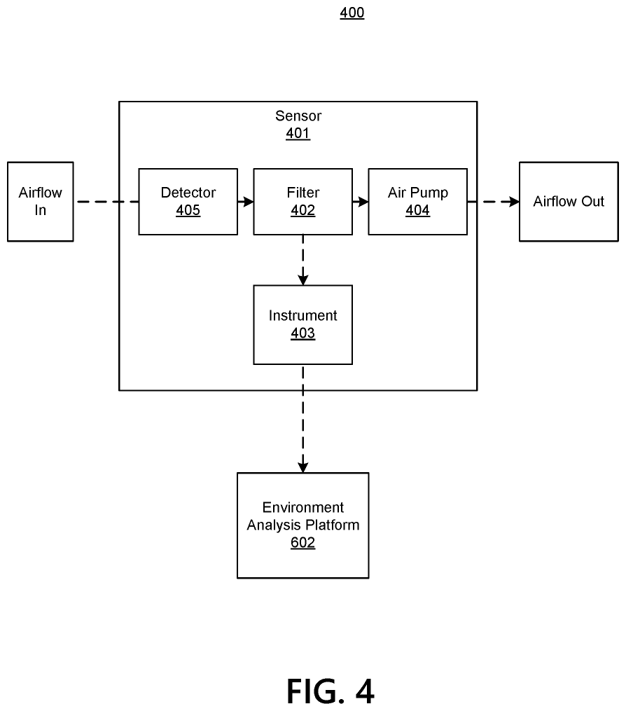

Additionally or alternatively, in some instances, the additional environment variables may include information indicating compositions of particles. For example, the environment analysis platform 602 may be configured to receive environment information from an electronic microscope. In this example, particles in the air of the environment being analyzed may be gathered (e.g., by a particle sensor equipped with a filter for catching particles, such as in system 400, which is illustrated at FIG. 4, and/or by other methods).

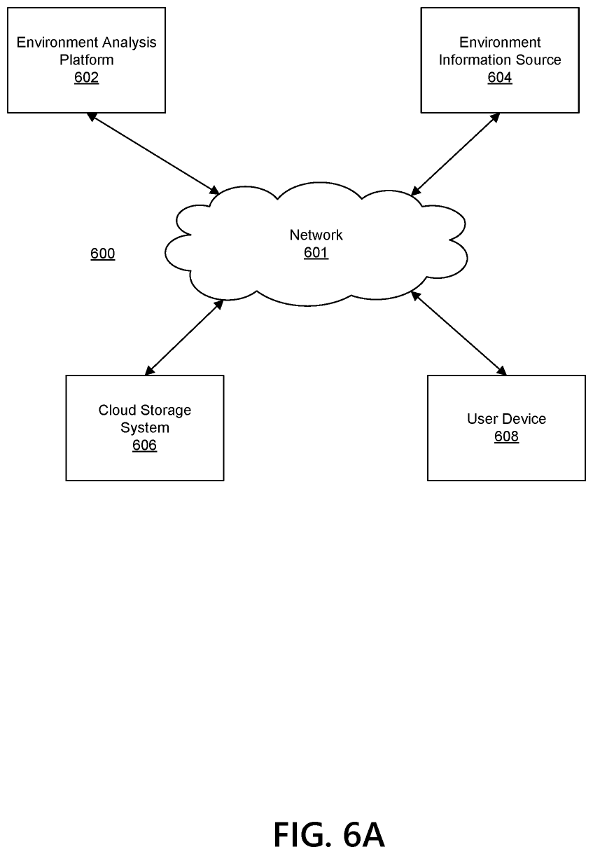

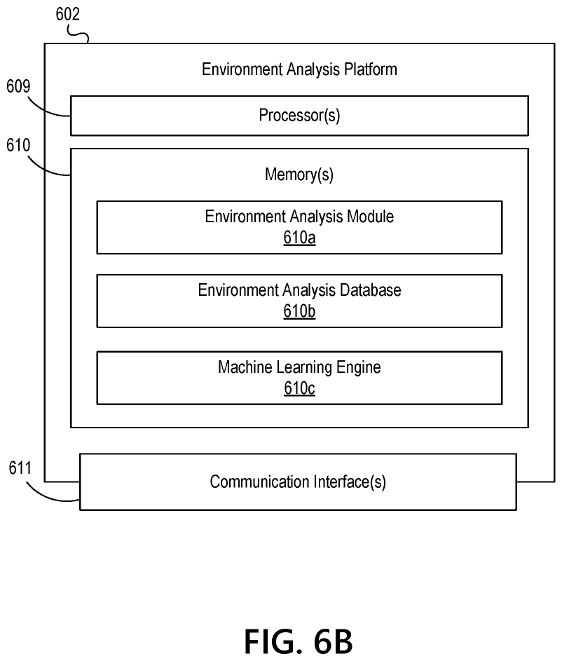

The environment analysis platform 602 may receive the environment information 204 from the environment information sources 202 via one or more wired or wireless data connections which may, e.g., be established via a communication interface communicatively coupled to the environment analysis platform 602 (e.g., communication interface 611, as illustrated in FIG. 6B, and/or other communication interfaces).

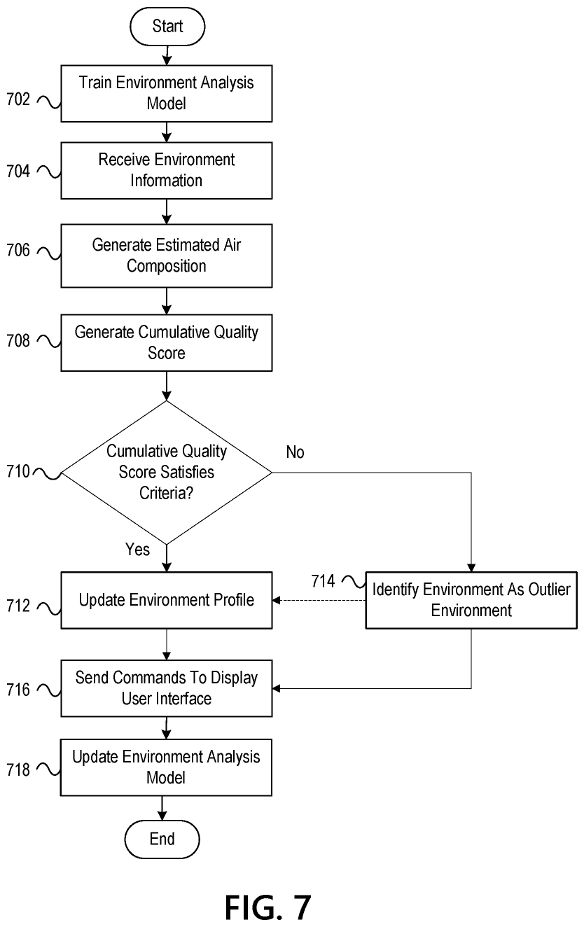

The environment analysis platform 602 may generate an estimated air composition 208 based on the environment information. For example, the environment analysis platform 602 may generate the estimated air composition that identifies specific particle types and/or concentrations of specific particle types based on the environmental information (e.g., particle size, particle count and/or other variables) using an environment analysis model (e.g., using the method illustrated in FIG. 7 and described below at step 706). The specific particle types may be and/or include one or more types of gas molecule (e.g., carbon dioxide molecules, carbon monoxide molecules, and/or other gas molecules), one or more types of particulate (e.g., aerosol particles, soot, tobacco smoke, smog, oil smoke, fly ash, and/or other particulates), one or more types of dust (e.g., cement dust, atmospheric dust, heavy dust, and/or other types of dust), one or more types of biological contaminant (e.g., pollen, mold spores, bacteria, viruses, and/or other biological contaminations) and/or other specific particle types. Creation of the environment analysis model may employ machine learning as illustrate in FIG. 2B.

FIG. 2B depicts an illustrative event flow for training an environment analysis model 210. Referring to FIG. 2B, environment analysis model training 209 may include training the environment analysis model 210 to generate an estimated air composition 213 based on environment information. In some examples, environment analysis platform 602 may train the environment analysis model 210. In other examples, the environment analysis model training 209 may be performed by another computing platform. In some instances, the environment analysis platform 602 may configure and/or otherwise train the environment analysis model based on historical sets of environment information 211 (e.g., wind speed, wind direction, gas concentration information, temperature, relative humidity, air pressure, dew point, vibration frequency, noise level, ventilation status, altitude, topographical information, occupancy, traffic counts (such as a historical number of motor vehicles passing through the environment in a given timeframe, a historical number of motor vehicles present in the environment at a specific time, a volume of traffic, the direction of traffic flow, vehicle type (e.g., cars, vans, hybrid models, electric models, diesel models, and/or other vehicle types) and/or other traffic counts), mold growth information, mildew particulate information, population information (such as the occupancy of a building, a number of individuals detected by a recording device, a number of individuals within a proximity to a sensor, a volume of individuals within a specified location, a number of individuals passing through an area in a specified time window, and/or other population counts), luminosity information (such as light intensity, one or more solar angles detected by a sensor, cloud cover levels, and/or other luminosity information), precipitation information (such as a volume of precipitation over a period of time, a type of precipitation (e.g., snow, rain, sleet, and/or other types of precipitation), and/or other precipitation information) and/or virus detection information) and historical estimated air compositions 212 corresponding to the historical sets of environment information 211. In some examples, the historical sets of environment information 211 may also include one or more particle counts, one or more particle concentrations, one or more particle sizes, one or more specific particles and particles types (such as the specific particles and particle types listed above), and/or other particle identification information. In some instances, to configure and/or otherwise train the environment analysis model 210, environment analysis platform 602 may process all (or a subset) of the historical sets of environment information 211 and historical estimated air compositions 212 by applying natural language processing, natural language understanding, supervised machine learning techniques (e.g., regression, classification, neural networks, support vector machines, random forest models, naïve Bayesian models, and/or other supervised techniques), unsupervised machine learning techniques (e.g., principal component analysis, hierarchical clustering, K-means clustering, and/or other unsupervised techniques), and/or other techniques. In doing so, the environment analysis platform 602 may train the environment analysis model 210 to generate, for a particular environment, an estimated air composition 213.

For example, in configuring and/or otherwise training the environment analysis model 210, environment analysis platform 602 may input historical sets of environment information 211 that environment analysis platform 602 has previously performed air composition estimation operations on, and historical estimated air compositions 212 generated while performing those operations, into the environment analysis model 210. For instance, the environment analysis platform 602 may use as a training set, a historical set of environment information 211 that corresponds to a particular historical estimated air composition 212. The environment analysis platform 602 may train the environment analysis model to minimize a difference between historical estimated air composition 212 and a generated estimated air composition 213. Sets of historical environment information 211 and corresponding historical estimated air compositions 212 may have been obtained by other environment analysis models and/or by manual collection and analysis, and may be based on different geographical locations, and/or different time periods of the same location. Additionally or alternatively, the environment analysis platform 602 may mine the historical sets of environment information and historical estimated air compositions to determine common estimated air compositions for specific environment information. For example, based on the historical sets of environment information and the corresponding historical estimated air compositions, the environment analysis platform 602 may identify that all historical sets of environment information including a particular particle size correspond to a particular estimated air composition. In another instance, the environment analysis platform 602 may identify a particular estimated air composition corresponding to a particular source of environment information, based on the historical sets of environment information and the corresponding historical estimated air compositions. For example, the environment analysis platform 602 may identify that multiple instances of a particular estimated air composition (or a portion of the particular estimated air composition, e.g., a specific particle type among a plurality of particle types) appear in the historical estimated air compositions and correspond to a common source of environment information (e.g., a specific particle sensor, a specific satellite, and/or other sources of environment information). In these instances, the environment analysis platform 602 may train the environment analysis model to generate the particular estimated air composition (or portion of the particular estimated air composition) for the particular source of environment information.

In some instances, to configure and/or otherwise train the environment analysis model, environment analysis platform 602 may update (e.g., continually) the environment analysis model 210 in order to improve the accuracy of the environment analysis model. For example, in configuring and/or otherwise training the environment analysis model 210, environment analysis platform 602 may, after using the environment analysis model to generate an estimated air composition 213, input the estimated air composition 213 and a known air composition 214 corresponding to the same environment into the environment analysis model. By inputting the estimated air composition into the model, the environment analysis platform 602 may create an iterative feedback loop that may continuously and dynamically update the environment analysis model to improve its accuracy. In these instances, the known air composition 214 may have been gathered and/or otherwise confirmed by a user of the environment analysis platform 602 (e.g., for the purpose of training the environment analysis model). For example, individuals (e.g., employees of an environment analysis enterprise, and/or other individuals) may gather and subsequently analyze the particles gathered by sensors (e.g., 101) using an instrument or process (e.g., electronic microscope, spectrum analyzer, chemical reaction, and/or other instruments or processes). In analyzing the particles, the instrument or process may identify the composition of the particles and/or other identifiable features of the particles. Information about the particles (e.g., the composition of the particles and/or other features of the particles) may be generated by the instrument or process (e.g., electronic microscope), and that information may be sent (e.g., by wireless data transfer) to the environment analysis platform 602 for input to the model as known air composition 214. In some instances, the known air composition 214 may be or include a historical estimated air composition confirmed by the user of environment analysis platform 602. The environment analysis model may generate, based on the estimated air composition 213and the known air composition 214, an error score indicating a margin of error between the estimated air composition and the known air composition.

In generating the error score, the environment analysis model may compare the estimated air composition 213 to the historical estimated air composition 212 and/or to the known air composition 214 in order to determine the margin of error. For example, the estimated air composition 213 and the known air composition 214 may be or include a number of identified particle types and particle counts corresponding to each of the identified particle types. The environment analysis model may compare the estimated number of identified particle types and particle counts to the known number of identified particle types and particle counts to determine the error score between the estimated air composition and the known air composition (e.g., by using root mean squared error calculations, mean squared error calculations, binary classification models, multiclass classification models, and/or other methods of calculating error). Based on the error score, the environment analysis platform 602 may update the environment analysis model to minimize the error. For example, the environment analysis platform 602 may identify whether or not the error score exceeds a threshold. Based on identifying that the error score does exceed the threshold, the environment analysis platform 602 may update the environment analysis model. In some examples of updating the environment analysis model, the environment analysis platform 602 may input additional historical environment information 211, corresponding historical estimated air compositions 213, and/or known air compositions 214 to configure and/or otherwise further train the environment analysis model (e.g., by using the methods described above) to more accurately estimate air compositions based on environment information.

FIG. 3 depicts an illustrative system 300 of gathering measured variables using a sensor for air composition estimation using an environment analysis model. Referring to FIG. 3, system 300 may include a sensor 301 connected (e.g., wired or wirelessly, permanently or momentarily) to the environment analysis platform 602 (e.g., for sending environment information). Sensor 301 may be a sensor device designed to generate environment information corresponding to an environment surrounding sensor 301 (e.g., a particle sensor designed to generate particle size and particle count measurements, and/or other sensor devices). Sensor 301 may generate the environment information by analyzing particles in air flowing through a detector 304 (e.g., by using cameras, lasers, and/or other methods of analyzing particles). The sensor 301 may be connected to the environment analysis platform 602 via a wired or wireless data connection (which may, e.g., be established via the communication interface 611). In some instances, the sensor 301 may be configured to receive airflow in from an environment and output airflow out from the sensor 301 after analyzing one or more particles captured while taking airflow in.

In some examples, sensor 301 may be further configured and/or otherwise constructed to include a filter 302. Filter 302 may be or include a filter medium (e.g., a surface media (such as a wire screen), a depth media (e.g., compressed felt), and/or other media). Filter 302 may capture particles from air flowing into sensor 301 using one or more particle capture methods (e.g., direct interception, inertial impaction, Brownian diffusion, hydrodynamic effects, electrostatic attraction, and/or other methods). Filter 302 may be installed and/or otherwise mechanically coupled with sensor 301 via a filter cartridge, and/or other coupling techniques. In some instances, filter 302 may be a reusable and/or multi-use filter. In some examples, filter 302 may be configured to be removed from sensor 301. In these examples, the filter 302 may be removed from the sensor 301 by a user of environment analysis platform 602 (e.g., an employee of an environment analysis enterprise, and/or other individuals) to evaluate the captured particles to confirm the accuracy of the environment information sent to environment analysis platform 602 by the sensor 301 (e.g., by analyzing the particles in a laboratory setting, by comparing the filter 302 to other filters, and/or by other methods). The evaluated information may be used as known air compositions 214 as discussed above with respect to FIG. 2B for training an environment analysis model.

Additionally or alternatively, sensor 301 may be further configured and/or otherwise constructed to include an air pump 303. Air pump 303 may be or include a device designed to generate airflow in (e.g., by operating a fan, a piston, and/or other methods of generating airflow) and produce airflow out. Air pump 303 may be installed and/or otherwise mechanically coupled to the sensor 301. Air pump 303 may operate continuously and/or operate on a configurable timer configurable by a user (e.g., an employee of an environment analysis enterprise, and/or other individuals). While detector 304, filter 302, and air pump 303 are illustrated in a particular order, these components can be arranged in any order, and detector 304 and filter 302 may be arranged adjacent or parallel to one another such that the air that flows through each is different.

FIG. 4 depicts another illustrative system of gathering particle composition information using a sensor for air composition estimation using an environment analysis model. Referring to FIG. 4, a system 400 may include a sensor 401 connected to the environment analysis platform 602 (e.g., for sending environment information). Sensor 401 may be a sensor device similar to sensor 301 and designed to generate environment information corresponding to an environment surrounding sensor 401 (e.g., a particle sensor designed to generate particle size and particle count measurements, and/or other sensor devices). Sensor 401 may generate the environment information by analyzing particles in air flowing through a detector 405 (e.g., by using cameras, lasers, and/or other methods of analyzing particles). The sensor 401 may be connected to the environment analysis platform 602 via a wired or wireless data connection (which may, e.g., be established via the communication interface 611). In some instances, the sensor 401 may be configured to receive airflow in from an environment and output airflow from the sensor 401 after analyzing one or more particles captured while taking airflow in.

In some examples, sensor 401 may be further configured and/or otherwise constructed to include a filter 402. Filter 402 may be or include a filter medium (e.g., a surface media (such as a wire screen), a depth media (e.g., compressed felt), and/or other media). Filter 402 may capture particles from air flowing into sensor 401 using one or more particle capture methods (e.g., direct interception, inertial impaction, Brownian diffusion, hydrodynamic effects, electrostatic attraction, and/or other methods). Filter 402 may be installed and/or otherwise mechanically coupled with sensor 401 via a filter cartridge, and/or other coupling techniques. In some instances, filter 402 may be a reusable and/or multi-use filter. In some examples, filter 402 may be configured to be removed from sensor 401. In these examples, the filter 402 may be removed from the sensor 401 by a user of environment analysis platform 602 (e.g., an employee of an environment analysis enterprise, and/or other individuals) to evaluate the captured particles to confirm the accuracy of the environment information sent to environment analysis platform 602 by the sensor 401 (e.g., by analyzing the particles in a laboratory setting, by using an electronic microscope, and/or by other methods). The evaluated information may be used as known air compositions 214 as discussed above with respect to FIG. 2b for training an environment analysis model.

In some instances, system 400 may additionally include an instrument 403 (e.g., spectrum analyzer, electronic microscope). Instrument 403 may be an electronic instrument which may, e.g., be low-powered and configured to analyze the particle composition of particles (e.g., by using transmission electron microscopy, scanning electron microscopy, spectrum analysis and/or other methods). In some examples, filter 402 my capture particles from the air flowing into sensor 401, which may be analyzed by instrument 403. In these examples, filter 402 may be mechanically coupled to instrument 403 and adapted to transfer the captured particles to the instrument. Based on analyzing the captured particles, instrument 403 may determine the composition of the captured particles. The sensor 401 may include the composition of the captured particles in the environment information sent to environment analysis platform 602.

Additionally or alternatively, sensor 401 may be further configured and/or otherwise constructed to include an air pump 404. Air pump 404 may be or include a device designed to generate airflow in (e.g., by operating a fan, a piston, and/or other methods of generating airflow) and produce airflow out. Air pump 404 may be installed and/or otherwise mechanically coupled to the sensor 401. Air pump 404 may operate continuously and/or operate on a configurable timer configurable by a user (e.g., an employee of an environment analysis enterprise, and/or other individuals). While detector 405, filter 402, instrument 403, and air pump 404 are illustrated in a particular order, these components can be arranged in any order, and detector 405 and filter 402 may be arranged adjacent or parallel to one another such that the air that flows through each is different. Additionally, detector 405 may not be included, or may be combined with instrument 403.

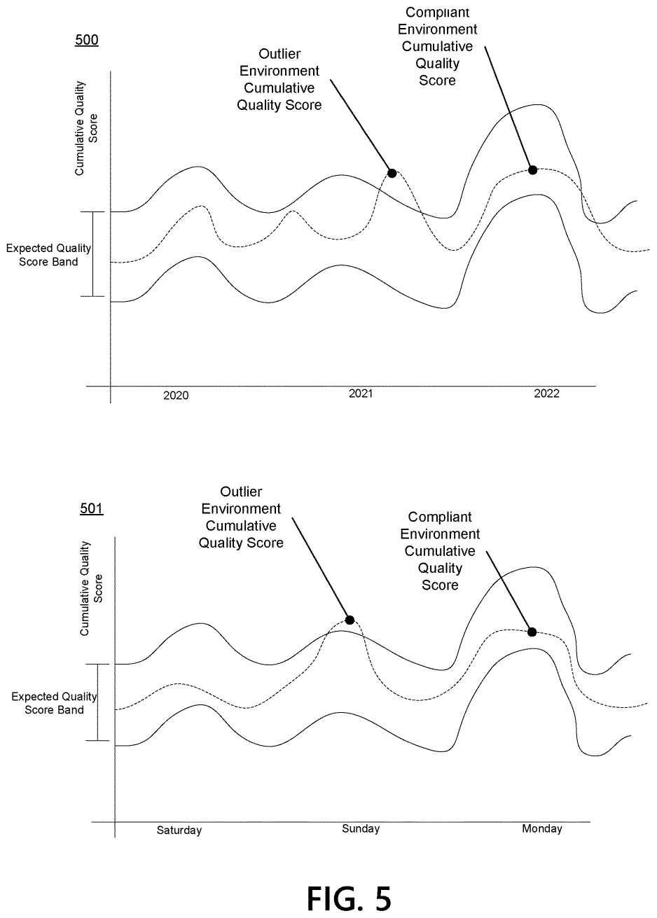

FIG. 5 depicts illustrative graphical user interfaces depicting particle score bands generated during air composition estimation (e.g., 200) using an environment analysis model (e.g., 210). The graphical user interfaces depicting particle score bands may be or include a particle score interface displayed on a user device (e.g., 608). The particle score interface may be displayed based on or in response to receiving one or more commands to display the particle score interface from environment analysis platform 602 (e.g., as described below at step 716). In some instances, the particle score interface may be a graphical user interface similar to annual particle score interface 500. Referring to FIG. 5, in some examples, the annual particle score interface 500 may include information depicting particle score bands. For example, the annual particle score interface 500 may include information such as a lower bound and an upper bound (e.g., solid lines) for an expected particle score band, one or more cumulative particle scores corresponding to particular environments (e.g., dashed line), and a graph plotting the lower bound and the upper bound for the expected particle score band and the one or more cumulative particle scores.

The lower bound and the upper bound for the expected particle score band may be generated by the environment analysis platform 602 during air composition estimation operations based on or in response to the environment analysis platform 602 receiving sets of additional environment information corresponding to an environment (e.g., historical environment information), where each of the sets of additional environment information further corresponds to a specific period of time (e.g., as described below at step 708). In some instances, the expected particle score band may represent a range indicating expected values for a cumulative particle score, where the cumulative particle score is generated by the environment analysis platform 602 (e.g., as described below at step 708) and corresponds to a particular environment. The range indicating the expected values for the cumulative particle score may indicate the expected value at a particular period of time. The one or more cumulative particle scores may indicate a cumulative particle score generated by the environment analysis platform 602 based on a set of environment information and corresponding to a particular environment at a particular time. For example, the one or more cumulative particle scores may indicate the cumulative particle score generated for the particular environment for a particular year (as shown by annual particle score interface 500). The graph plotting the lower bound and the upper bound for the expected particle score band and the one or more cumulative particle scores may include a y-axis representing values of cumulative particle scores and an x-axis representing the periods of time. The graph may further include indicators of the status of a cumulative particle score plotted on the graph, based on the location of the cumulative particle score. For example, the graph may indicate that a cumulative particle score corresponds to a compliant environment where the cumulative particle score is located within the expected particle score band for a particular time period. The graph may indicate that a cumulative particle score corresponds to an outlier environment where the cumulative particle score is located outside the expected particle score band for a particular time period. As shown in the figure, the cumulative particle score may be compliant during certain time periods, and non-compliant during other time periods.

Additionally or alternatively, in some instances, the particle score interface may be a graphical user interface similar to daily particle score interface 501. Referring again to FIG. 5, in some examples, the daily particle score interface 501 may include information depicting particle score bands. For example, daily particle score interface 501 may include information such as a lower bound and an upper bound for an expected particle score band, one or more cumulative particle scores corresponding to particular environments, and a graph plotting the lower bound and the upper bound for the expected particle score band and the one or more cumulative particle scores, in a similar manner to annual particle score interface 500. However, in these examples, the graph plotting the lower bound and the upper bound for the expected particle score band and the one or more cumulative particle scores may include an x-axis representing the periods of time different from the periods of time shown in annual particle score interface 500. For example, daily particle score interface 501 may include an x-axis representing days of the week.

In some examples, additional or alternative particle score interfaces may be displayed that may include x-axes representing different periods of time (e.g., hours, months, seasons, and/or other periods of time). Note that while the particle score interfaces illustrated in FIG. 5 depict a single estimated particle score band in the particle score interface, in some examples the particle score interfaces displayed by the user device may include one or more additional particle score bands generated by the environment analysis platform 602. Additionally, in some examples, the expected quality score band may be based on non-historical information, such as including simple limits of quality scores indicating that the environment does not pose a health risk to humans, other animals, and/or plants.

FIGS. 6A-6B depict an illustrative computing environment for air composition estimation using an environment analysis model in accordance with one or more example embodiments. Referring to FIG. 6A, computing environment 600 may include one or more computer systems. For example, computing environment 600 may include the environment analysis platform 602, an environment information source 604, a cloud storage system 606, and a user device 608.

Environment analysis platform 602 may be a computer system that includes one or more computing devices (e.g., servers, server blades, or the like) and/or other computer components (e.g., processors, memories, communication interfaces) that may be configured to operate, train and/or execute one or more machine learning models, such as environment analysis models. For example, the environment analysis platform 602 may train the one or more environment analysis models to generate estimated air compositions for one or more environments (e.g., as described above and illustrated at FIG. 2B). In some instances, environment analysis platform 602 may be controlled or otherwise maintained by an environment analysis organization such as a financial institution. Although shown as an independent environment analysis platform 602, in some instances, the environment analysis platform 602 may be part of and/or otherwise integrated into the user device 608 without departing from the scope of the disclosure.

Environment information source 604 may be and/or otherwise include one or more computing devices (e.g., servers, server blades, and/or other devices) and/or other computer components (e.g., processors, memories, communication interfaces) that may be configured to gather environment information corresponding to a particular environment and send the environment information to the environment analysis platform 602. Although shown as an independent information source, in some instances, the environment information source 604 may be or include one or more information sources, such as sensors 101, environment information sources 202, sensor 301, and/or sensor 401. The environment information source 604 may be or include one or more of: a particle sensor, a network of sensors located at different geographical locations, a repository of information gathered by multiple unassociated sensors, satellites gathering environmental information, a repository of automotive traffic information, one or more devices gathering environment information of the interior of a building, one or more devices gathering environment information from a moving vehicle, one or more anemometers, a database corresponding to an environment analysis organization, and/or other information sources.

Cloud storage system 606 may be and/or otherwise include one or more computing devices (e.g., servers, server blades, and/or other devices) and/or other computer components (e.g., processors, memories, communication interfaces) that may be configured to create, host, modify, and/or otherwise validate a cloud-based storage system (e.g., a distributed ledger). The cloud storage system 606 may be synchronized across multiple nodes (e.g., sites, institutions, geographical locations, and/or other nodes) and may be accessible by multiple users (who may, e.g., be employees or clients of an environment analysis organization). The data stored at the databases of cloud storage system 606 may include any of the data (e.g., environment information, and/or other information) stored at and/or created by environment analysis platform 602 and/or any additional data. In some instances, the databases stored on cloud storage system 606 may be accessed by, validated by, and/or modified by any of, user device 608, environment analysis platform 602, and/or other devices.

User device 608 may be a computing device (e.g., laptop computer, desktop computer, mobile device, tablet, smartphone, server, server blade, and/or other device) and/or other data storing or computing component (e.g., processors, memories, communication interfaces, databases) that may be configured to transfer data between users and/or perform other user functions (e.g., receiving display commands, and/or other functions). In one or more instances, user device 608 may correspond to a user account (which may, e.g., be associated with an employee of the environment analysis organization, may be a client of the environment analysis organization, may be otherwise associated with the enterprise organization, or the like). In one or more instances, the user device 608 may be configured to communicate with one or more systems (e.g., environment analysis platform 602, environment information source 604, and/or other systems) to perform a data transfer, receive display commands, and/or to perform other functions.

Computing environment 600 also may include one or more networks, which may interconnect environment analysis platform 602, environment information source 604, cloud storage system 606, user device 608. For example, computing environment 600 may include a network 601 (which may interconnect, e.g., environment analysis platform 602, environment information source 604, cloud storage system 606, and user device 608).

In one or more arrangements, environment analysis platform 602, environment information source 604, cloud storage system 606, user device 608 may be any type of computing device capable of sending and/or receiving requests and processing the requests accordingly. For example, environment analysis platform 602, environment information source 604, cloud storage system 606, user device 608, and/or the other systems included in computing environment 600 may, in some instances, be and/or include server computers, desktop computers, laptop computers, tablet computers, smart phones, or the like that may include one or more processors, memories, communication interfaces, storage devices, and/or other components. As noted above, and as illustrated in greater detail below, any and/or all of environment analysis platform 602, environment information source 604, cloud storage system 606, user device 608, may, in some instances, be special-purpose computing devices configured to perform specific functions. For example, each may include memory that includes computer readable instructions that, when executed by a processor, cause the special-purpose computing device to perform one or more functions described herein.

Referring to FIG. 6B, environment analysis platform 602 may include one or more processors 609, memory 610, and communication interface 611. A data bus may interconnect processor 609, memory 610, and communication interface 611. Communication interface 611 may be a network interface configured to support communication between environment analysis platform 602 and one or more networks (e.g., network 601, or the like). Communication interface 611 may be communicatively coupled to the processor 609. Communication interface 611 may be configured to receive sets of environment information from one or more environment information sources via one or more wireless data connections. Memory 610 may include one or more program modules having instructions that, when executed by processor 609, cause environment analysis platform 602 to perform one or more functions described herein and/or one or more databases that may store and/or otherwise maintain information which may be used by such program modules and/or processor 609. In some instances, the one or more program modules and/or databases may be stored by and/or maintained in different memory units of environment analysis platform 602 and/or by different computing devices that may form and/or otherwise make up environment analysis platform 602. For example, memory 610 may have, host, store, and/or include environment analysis module 610a, environment analysis database 610b, and machine learning engine 610c.

Environment analysis module 610a may have instructions that direct and/or environment analysis platform 602 to perform environment analysis (e.g., as described with respect to FIG. 2A). Environment analysis database 610b may have instructions causing environment analysis platform 602 to store environment information, estimated air compositions, and/or cumulative particle scores (that may, e.g., be used to perform environment analysis). Machine learning engine 610c may contain or include instructions causing environment analysis platform 602 to train, implement, and/or update an environment analysis model (e.g., as described with respect to FIG. 2B). In some instances, machine learning engine 610c may be used by environment analysis platform 602 and/or environment analysis module 610a to refine and/or otherwise update methods for environment analysis, and/or other methods described herein.

FIG. 7 depicts an illustrative method for air composition estimation using an environment analysis model in accordance with one or more example embodiments. Referring to FIG. 7, at step 702, the environment analysis platform 602 may train the environment analysis model. In training the environment analysis model, the environment analysis platform 602 may configure and/or otherwise train the environment analysis model to generate an estimated air composition based on environment information. For example, the environment analysis platform 602 may configure and/or otherwise train the environment analysis model using the methods illustrated in FIG. 2B described above in environment analysis model training 209, and/or other training methods.

At step 704, the environment analysis platform 602 may receive environment information. For example, the environment analysis platform 602 may receive the environment information in the form of a set of environment information from the environment information source 604. The environment analysis platform 602 may receive the set of environment information via a wired or wireless data connection established between the environment information source 604 and the communication interface 611. In some instances, the environment analysis platform 602 may additionally or alternatively receive the set of environment information indirectly. For instance, the environment analysis platform 602 may receive the set of environment information from a repository (e.g., a web-based server, database, cloud storage system, and/or other repositories), which may for example be implemented in cloud storage system 606. In these examples, the environment analysis platform 602 may retrieve the set of environment information from the repository (e.g., via webscraping methods, application programming interface (API) calls, information transfer requests, and/or other methods).

The set of environment information may include one or more measured variables corresponding to one or more known attributes of a known particle, and/or one or more additional environment variables. The one or more additional environment variables may include one or more of: wind speed, wind direction, particle concentrations, gas concentrations, temperature information, relative humidity information, air pressure information, dew point information, vibration information, noise information, environment ventilation status, altitude information, topographical information, occupancy information, traffic counts, mold growth information, mildew growth information, population information, luminosity information, precipitation information, and/or virus detection information. In some instances, the environment analysis platform 602 may receive additional sets of environment information from the environment information source 604. In these instances, each of the sets of additional environment information may correspond to a specific period of time. For example, the environment analysis platform 602 may receive a set of environment information corresponding to Monday, a set of information corresponding to Tuesday, a set of information corresponding to a specific week of the year, a set of information corresponding to a specific month, and so on.

At step 706, the environment analysis platform 602 may generate, using the environment analysis model, the estimated air composition for the environment. In generating the estimated air composition, the environment analysis platform 602 may input the set of environment information into the environment analysis model. The environment analysis model may use the set of environment information to estimate one or more types of particle present in the environment (e.g., one or more gas molecules (e.g., carbon dioxide molecules, carbon monoxide molecules, and/or other gas molecules), one or more particulates (e.g., aerosol particles, soot, tobacco smoke, smog, oil smoke, fly ash, and/or other particulates), one or more types of dust (e.g., cement dust, atmospheric dust, heavy dust, and/or other types of dust), one or more biological contaminants (e.g., pollen, mold spores, mildew spores, bacteria, viruses, and/or other biological contaminations) and/or other types of particle). In some instances, the estimated air composition may be based wholly or in part on one or more particle sizes (e.g., measured by sensor 101 or 301) included in the set of environment information. For example, inputting a specific particle size may cause the environment analysis model to generate an estimated air composition identifying a type of particle corresponding to the specific particle size. Additionally or alternatively, the estimated air composition may be based wholly or in part on one or more particle counts (e.g., measured by sensor 101 or 301) included in the set of environment information. For example, inputting a specific particle count may cause the environment analysis model to generate an estimated air composition identifying a type of particle corresponding to the specific particle count (e.g., based on a stored correlation between the specific particle count and historical estimated air compositions). Additionally or alternatively, in some examples, other environment variables may be used to generate the estimated air composition. In some examples, the environment analysis model may associate a particular particle size and/or particle count with different types of particles, depending upon the additional environment variables (e.g. temperature, humidity, season, time of day, etc.). In some instances, estimated air composition may be based wholly or in part on one or more variables. For example, inputting a current air pressure, current relative humidity, current wind speed/direction, and/or other current variables may cause the environment analysis model to generate an estimated air composition identifying a type of particle corresponding to one or more current variables of the additional environment variables. For instance, based on a stored correlation between a current humidity and historical information (e.g., historical particle counts, historical particles sizes, historical humidity, etc. (which may, e.g., have previously been used to train the environment analysis model), the environment analysis model may generate an estimated air composition identifying at least one particle type associated with the historical humidity. The estimated air composition may be or include one or more identified types of particles and corresponding particle counts.

In some examples, the environment analysis platform 602 may have previously trained the environment analysis model to employ an environment analysis algorithm to determine the estimated air composition. In some instances, the environment analysis algorithm may use some or all of the information included in the set of environment information. In using the environment analysis algorithm, the environment analysis platform 602 may use some or all of the information in one or more historical sets of environment information to identify currently unidentified particle types from the set of environment information. For example, the environment analysis platform 602 may execute the environment analysis algorithm using the following constraints/parameters:
If:
   (Historical Particle 1 size=Unidentified Particle 1 size),
   and
   (Historical Particle 1 vibration frequency=Unidentified Particle 1 vibration frequency),
   and
   (Historical air pressure=current air pressure);
then

### Unidentified Particle 1=Historical Particle 1.

In this example, based on identifying that unidentified particle 1 is equivalent to historical particle 1, then the environment analysis platform 602 may generate an estimated air composition including historical particle 1. Here, “equivalent” was formulated to consider size, and vibration frequency at a common air pressure, but different or additional variables could be included in the determination. Additionally or alternatively, the environment analysis platform 602 may execute the environment analysis algorithm using one or more additional constrains/parameters, which may, e.g., include one or more additional environment variables.

In some instances, after generating the estimated air composition, the environment analysis platform 602 may configure an environment profile corresponding to the environment. For example, the environment analysis platform 602 may configure the environment profile to maintain a record of the environment. The record of the environment may be or include geographic information of the environment (e.g., a geographic location, topography, and/or other geographic information), the estimated air composition of the environment, one or more historical estimated air compositions of the environment, an environmental status of the environment (e.g., an indicator of whether the environment is a compliant environment or an outlier environment), and/or other information. The environment analysis platform 602 may maintain the record by storing the environment profile at a storage system, for example in internal memory of the environment analysis platform 602, and/or in an external memory, such as cloud storage system 606.

At step 708, the environment analysis platform 602 may generate a cumulative particle score based on the estimated air composition. In generating the cumulative particle score, the environment analysis platform 602 may generate the cumulative particle score as an integer value, alphanumeric character, percentage value, decimal value, hexadecimal value, binary value, and/or other representations of the cumulative particle score. The cumulative particle score may indicate a measure of air quality in the environment corresponding to the set of environment information. For example, a first environment corresponding to a higher cumulative particle score than a second environment may contain a higher concentration of particles than the second environment. In generating the cumulative particle score, the environment analysis platform 602 may analyze the particle types and corresponding particle counts included in the estimated air composition. For example, in some instances the environment analysis platform 602 may generate a cumulative particle score equivalent to the sum of the particle counts corresponding to each particle type. In another example, in generating the cumulative particle score, the environment analysis platform 602 may assign weight coefficients to specific particle types (e.g., indicating more harmful or more benign particles). The environment analysis platform 602 may analyze estimated air composition to determine the identified particle types and the corresponding particle counts. The environment analysis platform 602 may multiply the particle counts corresponding to each specific identified particle type by the respective assigned weight coefficient to generate weighted values. The environment analysis platform 602 may compute the cumulative particle score by calculating the sum of the weighted values. For example, the environment analysis platform 602 may calculate the cumulative particle score by executing the following formula:
Cumulative Quality Score=
   (Particle A Coefficient*Particle A Count)+
   (Particle B Coefficient*Particle B Count)+

(Particle C Coefficient*Particle C Count). In some instances, the environment analysis platform 602 may automatically assign the weight coefficients based on predetermined settings. For example, the environment analysis platform 602 may include predetermined settings assigning weight coefficients based on a consideration of danger of the particle types (e.g., particle types known to damage human lungs may be assigned higher weight coefficients than particle types that are not harmful to human lungs, and/or other considerations of danger). In some examples, the environment analysis platform 602 may additionally or alternatively assign the weight coefficients based on user input (e.g., user input received from an employee of an environment analysis organization, and/or other individuals).

In some examples, the environment analysis platform 602 may additionally have received sets of additional environment information from the environment information source 604 (e.g., as described above at step 704). Each of the sets of additional environment information may correspond to a specific period of time (e.g., a specific day of the week, and/or other periods of time). In some instances, one or more of the sets of additional environment information may correspond to the same environment as the set of environment information received at step 704. Additionally or alternatively, one or more of the sets of additional environment information may correspond to a different environment that may, e.g., possess similar characteristics (e.g., humidity level, air pressure, climate, and/or other characteristics) to the environment corresponding to the set of environment information.

Based on the sets of additional environment information and the set of environment information, the environment analysis platform 602 may generate one or more expected particle score bands for a particular environment. In generating the one or more expected particle score bands, the environment analysis platform 602 may analyze the sets of additional environment information and the set of environment information to determine an upper bound and a lower bound for each of the one or more expected particle score bands. For example, the environment analysis platform 602 may have received five sets of additional environment information, where the five sets of additional environment information each correspond to one of the previous five Fridays. In this example, based on the five sets of additional environment information, the environment analysis platform 602 may determine a maximum and a minimum cumulative particle score for the previous five Fridays (e.g., by using the environment analysis model to generate estimated air compositions and generating corresponding cumulative particle scores, as described above at steps 706-708). The environment analysis platform 602 may use the maximum and minimum cumulative particle scores as the upper bound and lower bound of the expected particle score band, respectively. Although the above example describes generating a cumulative particle score band for a specific day of the week, in some examples the environment analysis platform 602 may generate one or more cumulative particle score bands corresponding to different periods of time (e.g., months, weeks, years, seasons, and/or other periods of time) and/or generate one or more cumulative particle score bands corresponding to multiple specific periods of time (e.g., each day of the week, each month of the year, each season of the year, and/or other specific

### Periods of Time).

In some instances, the one or more expected particle score bands may represent ranges indicating expected values for a cumulative particle score corresponding to a particular environment. In some examples, the environment analysis platform 602 may plot the one or more expected quality score bands on a graph that may, e.g., be displayed to a user via a graphical user interface (e.g., as illustrated at FIG. 5 and described above).

At step 710, the environment analysis platform 602 may determine whether or not the cumulative particle score satisfies a criteria. The criteria may be or include a threshold value, a threshold range, a limit on the particle count of one or more particle types, and/or other criteria. The criteria may indicate whether the environment is a compliant environment or an outlier environment. In determining whether or not the cumulative particle score satisfies the criteria, the environment analysis platform 602 may compare the cumulative quality score to the criteria. For example, the cumulative quality score may have or be a numerical value of 140, and the criteria may have or be a threshold value of 100, where cumulative particle scores below the threshold value satisfy the criteria. Based on the comparison, the environment analysis platform 602 may determine that the cumulative quality score exceeds the threshold value and thus fails to satisfy the criteria, which may, e.g., indicate that the corresponding environment is an outlier environment.

In another instance, the cumulative quality score may be or include a sum of particle counts corresponding to specific particle types, and the criteria may be a limit on a particle count corresponding to a specific particle, where particle counts corresponding to the specific particle and exceeding the limit fail to satisfy the criteria. In this instance, a cumulative quality score including a particle count of 50 micrograms per cubic meter of Particle Type A would fail to satisfy a criteria imposing a limit of 35 micrograms per cubic meter on Particle Type A. In another example, the criteria may be or include one or more expected particle score bands (e.g., the one or more expected particle score bands generated and described above at step 708). In determining whether the cumulative particle score satisfies the criteria, the environment analysis platform 602 may determine whether or not the cumulative particle score is within the one or more expected particle score bands. For example, a cumulative particle score corresponding to a specific period of time, that is or includes a value outside of the expected quality score band corresponding to the specific period of time, may fail to satisfy the criteria.

Based on or in response to determining that the cumulative particle score satisfies the criteria, the environment analysis platform 602 may proceed to update the environment profile (e.g., as described below at step 712). Based on or in response to determining that the cumulative particle score fails to satisfy the criteria, the environment analysis platform 602 may instead identify the environment corresponding to the cumulative particle score as an outlier environment (e.g., as described below at step 714).

At step 712, based on or in response to determining that the cumulative particle score satisfies the criteria, the environment analysis platform 602 may proceed to update the environment profile (e.g., the environment profile configured at step 706, as described above). In updating the environment profile, the environment analysis platform 602 may modify or confirm the environment status of the environment based on the determination. For example, if the environment profile includes an environment status indicating that the environment is a compliant environment, the environment analysis platform 602 may confirm the environment status. If the environment profile includes an environment status indicating that the environment is an outlier environment, the environment analysis platform 602 may modify the environment status to indicate that the environment is now a compliant environment. Based on or in response to updating the environment profile, the environment analysis platform 602 may proceed to send one or more commands to display a user interface (e.g., as described below at step 716).

At step 714, based on or in response to determining that the cumulative particle score fails to satisfy the criteria, the environment analysis platform 602 may identify the environment corresponding to the cumulative particle score as an outlier environment. Based on or in response to identifying the environment as an outlier environment, the environment analysis platform 602 may generate one or more hazard notifications. The hazard notifications may indicate the cumulative particle score's failure to satisfy the criteria and the estimated air composition in order to alert individuals or organizations of a potential environmental hazard located in the environment. In some instances, based on identifying the environment as an outlier environment, the environment analysis platform 602 may additionally update the environment profile (e.g., via step 712). In updating the environment profile, the environment analysis platform 602 may modify or confirm the environment status of the environment based on the determination. For example, if the environment profile includes an environment status indicating that the environment is an outlier environment, the environment analysis platform 602 may confirm the environment status. If the environment profile includes an environment status indicating that the environment is a compliant environment, the environment analysis platform 602 may modify the environment status to indicate that the environment is now an outlier environment. Based on or in response to identifying the environment as an outlier environment, the environment analysis platform 602 may proceed to send one or more commands to display a user interface (e.g., as described below at step 716).

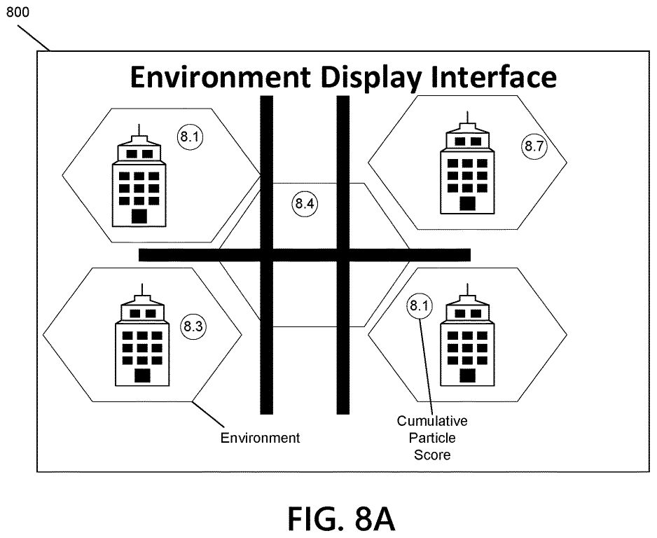

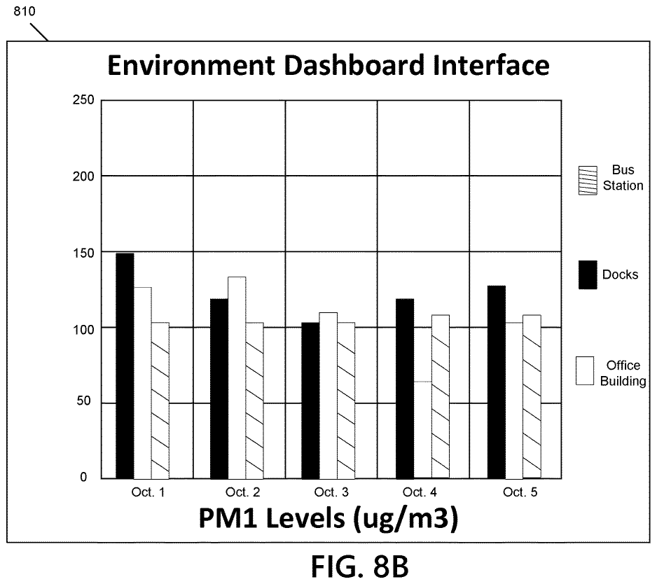

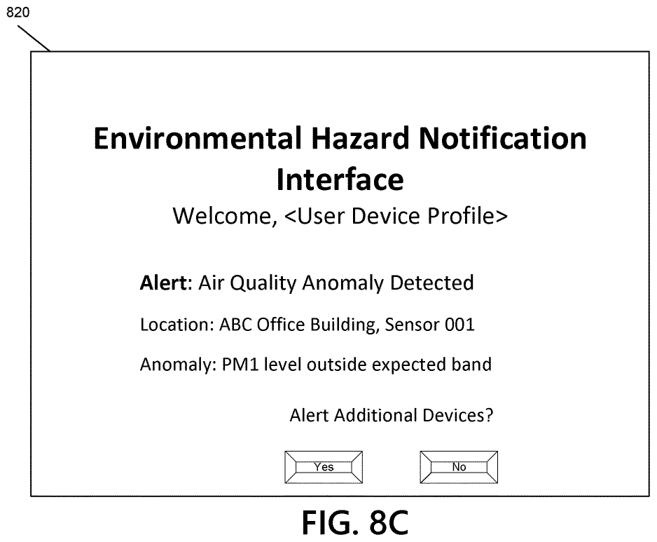

At step 716, the environment analysis platform 602 may send one or more commands to display a user interface to user device 608. For example, the environment analysis platform 602 may send the one or more commands to user device 608 via the communication interface 611 while a wired or wireless data connection is established. The one or more commands to display the user interface may cause user device 608 to display the user interface. For example, in the user device 608 may display an environment display interface. In displaying an environment display interface, the user device 608 may display a graphical user interface similar to environment display interface 800, which is illustrated in FIG. 8A. Referring to FIG. 8A, in some instances, the environment display interface 800 may include information corresponding to the environment and the environment analysis operations performed by the environment analysis platform 602. For example, the environment display interface 800 may include information such as a map of a geographical region (e.g., a city grid map, a three-dimensional city map, and/or other geographical maps), one or more environments the environment analysis platform 602 has performed environment analysis operations on, one or more cumulative particle scores corresponding to the one or more environments, and/or other information.

Additionally or alternatively, based on or in response to the one or more commands to display the user interface, the user device 608 may display an environment dashboard interface. For example, in displaying the environment dashboard interface, the user device 608 may display a graphical user interface similar to environment dashboard interface 810, which is illustrated in FIG. 8B. The environment dashboard interface 810 may correspond to the environment. Referring to FIG. 8B, in some instances, the environment dashboard interface 810 may include information corresponding to environment. For example, the environment dashboard interface 810 may include information such as a representation based on the estimated air composition corresponding to the environment, and/or other information. For example, the representation may be or include a graph, which may, e.g., display the particle counts for one or more particle types identified by the environment analysis platform 602 and included in the estimated air composition. In these examples, the graph may display the particle counts over a period of time (e.g., days, weeks, months, and/or other periods of time).

Additionally or alternatively, based on or in response to the one or more commands to display the user interface, the user device 608 may display a particle score interface. For example, in displaying the particle score interface, the user device 608 may display a graphical user interface similar to annual particle score interface 500 and/or daily particle score interface 501, which are illustrated in FIG. 5. The user device 608 may display the particle score interface as illustrated in FIG. 5 and described above.

In some instances, based on the determination of whether or not the cumulative particle score satisfies the criteria (e.g., as described above at step 710), the environment analysis platform 602 may send one or more commands to display a user interface indicating a result of the determination to user device 608. Based on or in response to the one or more commands to display the user interface, the user device 608 may display an environmental hazard notification interface. In some instances, the environmental hazard notification interface may be configured to receive user input through the user device 608. For example, in displaying the environmental hazard notification interface, the user device 608 may display a graphical user interface similar to environmental hazard notification interface 820, which is illustrated in FIG. 8C. Referring to FIG. 8C, the environmental hazard notification interface 820 may include information corresponding to the determination. For example, the environmental hazard notification interface 820 may include information such as an alert indicating the status of the environment (e.g., a compliant environment, an outlier environment, and/or other statuses), one or more locations of the environment information source (e.g., environment information source 604) that provided sets of environment information (e.g., the set of environment information), and one or more descriptions of the determination (e.g., a description of the criteria and why the cumulative particle score failed to satisfy the criteria, and/or other descriptions). In some instances, based on a determination that the cumulative particle score fails to satisfy the criteria, the environmental hazard notification interface 820 may additionally or alternatively include one or more hazard notifications (e.g., the hazard notifications generated at step 714, as described above).

The environmental hazard notification interface 820 may also display input mechanisms or selectable options requesting user input. For example, the environmental hazard notification interface 820 may display one or more of: a button or buttons, toggle or toggles, check box or boxes, and/or other interface elements. For example, as illustrated in FIG. 8C, the input mechanisms may be buttons the user can select to decide whether or not to alert additional devices. In some instances, based on user input directing the user device 608 to alert additional devices, the user device 608 may send one or more commands directing the environment analysis platform 602 to alert additional devices. In these instances, the one or more commands directing the environment analysis platform 602 to alert additional devices may cause the environment analysis platform 602 to send an environmental hazard notification interface to one or more additional user devices (e.g., one or more environment information sources (such as environment information source 604), devices corresponding to clients of an environment analysis organization, devices corresponding to employees of an environment analysis organization, devices corresponding to employees of an environmental alert service, and/or the like).

For example, in some instances the environment information source 604 may be or include one or more non-static devices (e.g., mobile communication devices, wearable devices, and/or other devices). In these instances, based on user input directing the environment analysis platform 602 to alert additional devices, the environment analysis platform 602 may send an environmental hazard notification interface to the one or more non-static devices. In some instances, the one or more non-static devices may notify the user of the environmental hazard notification interface. For example, the one or more non-static devices may be or include a wearable device (e.g., a band, smart watch, smart clothing, smart bracelet, fitness tracker, smart belt, and/or other wearable devices) configured to produce a haptic response (e.g., a vibration, buzz, pulse, and/or other haptic responses), an audio response (e.g., music, speech, storytelling), and/or a text response based on receiving the environmental hazard notification interface.

In some instances, in alerting the additional devices, the environment analysis platform 602 may alert list of mobile devices corresponding to a specific geographic location. The environment analysis platform 602 may have previously received a list of all the mobile devices present in the specific geographic location and configured to receive an environmental hazard notification interface. For example, the environment analysis platform 602 may have received a list of mobile devices corresponding to users who subscribe to a service provided by an environment analysis organization managing the environment analysis platform 602, and/or other lists. The environment analysis platform 602 may have received the list via the communication interface 611 and while a wireless data connection was established. The environment analysis platform 602 may send one or more commands directing each mobile device of the list of mobile devices corresponding to the specific geographic location to display a user interface (e.g., an environmental hazard notification interface, and/or other interfaces). In some instances, the specific geographic location may be a configurable location configured by the user of the environment analysis platform 602. For example, a user (e.g., an employee of an environment analysis organization, and/or other users) may configure the specific geographic location to encompass one or more environments on which the environment analysis platform 602 performs environment analysis operations.

Referring back to FIG. 7, at step 718, the environment analysis platform 602 may update the environment analysis model. In updating the environment analysis model, the environment analysis platform 602 may refine and/or otherwise update the environment analysis model to improve the accuracy in generating an estimated air composition based on environment information. For example, the environment analysis platform 602 may refine and/or otherwise update the environment analysis model as described above with respect to FIG. 2B with environment analysis model training 209, and/or other updating methods.

The environment analysis platform 602 may perform additional environment analysis operations to those included in the method illustrated in FIG. 7 and described above. In some instances, the environment analysis platform 602 may determine an age of environment information source 604 (which may, e.g., be a particle sensor, and/or other source of environment information) based on the set of environment information. In determining the age of the environment information source 604, the environment analysis platform 602 may compare the set of environment information gathered by the environment information source 604 to one or more historical sets environment information and/or known environment information. For example, the environment analysis platform 602 may determine that a particle count included in the set of environment information differs from a particle count included in one or more historical sets of environment information corresponding to the same environment by a specific amount. Based on a determination that the particle count included in the set of environment information should match the particle count included in the one or more historical sets of environment information (e.g., by analyzing the particle count included in current known environment information, which may have been gathered by a different environment information source), the environment analysis platform 602 may identify that the accuracy of environment information source 604 has degraded to or below a particular accuracy threshold. The environment analysis platform 602 may determine the age of the environment information source 604 by comparing the particular accuracy threshold to a list of accuracy thresholds corresponding to specific ages of environment information sources.

In some instances, based on determining the age of the environment information source 604, the environment analysis platform 602 may cause the environment information source 604 to recalibrate in order to improve the accuracy of environment information source 604. For example, the environment analysis platform 602 may send a notification to a user (e.g., an employee of an environment analysis organization managing environment information source 604, and/or other users) directing the user to recalibrate environment information source 604.

In performing additional environment analysis operations, the environment analysis platform 602 may additionally or alternatively determine a source of one or more particles identified in the estimated air composition. In some instances, the environment analysis platform 602 may determine the source of the one or more particles concurrent with and/or after generating the estimated air composition (e.g., as described at step 706 above). In identifying the source of the one or more particles, the environment analysis platform 602 may analyze a subset of the set of environment information in order to determine a source location corresponding to the source of the one or more particles identified in the estimated air composition. The environment analysis platform 602 may analyze the particle size, particle weight, particle movement information (e.g., movement direction, movement speed, and/or other movement information), wind speed, wind direction, and/or other environment information. For example, the environment analysis platform 602 may compare the specific particle size, specific particle weight, and specific particle movement speed of a specific particle with the wind speed and wind direction and determine, based on the comparison a source location from which a particle corresponding to the specific particle size, specific particle weight, and specific particle movement speed could have traveled based on the wind speed and wind direction.

Based on determining the source location, the environment analysis platform 602 may determine the source of the one or more particles based on geographic information. The geographic information may be retrieved from cloud storage system 606 and/or other information storage systems. In determining the source of the one or more particles based on geographic information, the environment analysis platform 602 may compare the source location with the geographic information. For example, based on geographic information indicating the presence of a particular structure (e.g., a power plant, refinery, and/or other structure) at or near the source location, the environment analysis platform 602 may identify the particular structure as the source of the one or more particles identified in the estimated air composition.

Based on determining the source of the one or more particles identified in the estimated air composition, the environment analysis platform 602 may update the estimated air composition generated at step 706. In updating the estimated air composition, the environment analysis platform 602 may update (e.g. by identifying, reclassifying, and/or otherwise updating) one or more identified particle types included in the estimated air composition. For example, based on determining the source of the one or more particles identified in the estimated air composition to be a particular structure (e.g., a power plant, refinery, and/or other structure), the environment analysis platform 602 may reclassify one or more identified particle types as a different particle type associated with the particular structure.

Additionally or alternatively, in performing additional environment analysis operations, the environment analysis platform 602 may cause, based on the determination of whether or not the cumulative particle score satisfies the criteria (e.g., as described at step 710 above) one or more environment safety actions to occur. For example, in some instances the environment analysis platform 602 may be deployed as part of a system (e.g., an environment control system, and/or other systems). In these instances, the environment analysis platform 602 may cause the activation of one or more additional devices within the system. For example, the environment analysis platform 602 may cause the activation of an air filtration device which may, e.g., be deployed and/or otherwise installed in the same location as the environment information source 604. The environment analysis platform 602 may cause the activation of the one or more additional devices within the system by sending one or more commands (e.g., via the communication interface 611) to the one or more additional devices within the system.

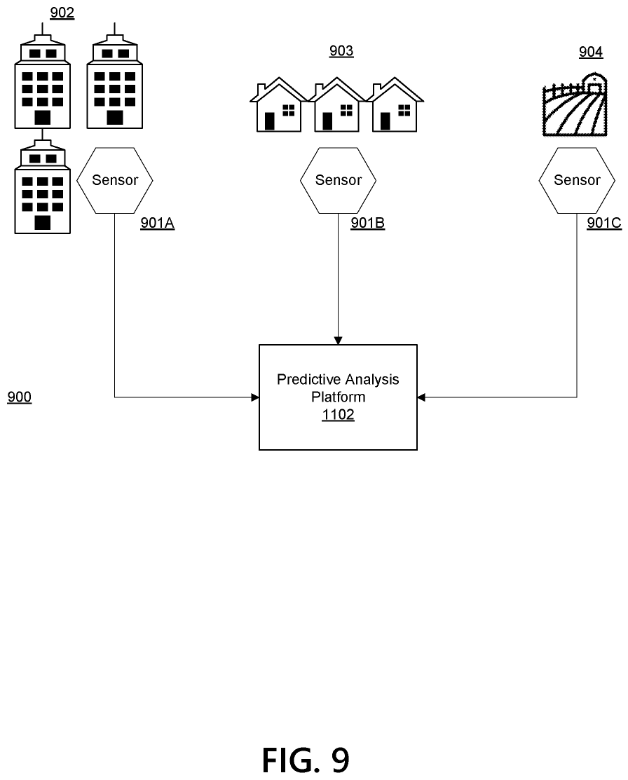

FIG. 9 depicts an illustrative environment information network for gathering measurement information corresponding to an environment and sending the information to a computing platform (e.g., a predictive analysis platform, such as predictive analysis platform 1102) for predictive analysis. In some instances, the environment depicted in FIG. 9 may be the same environment as described with respect to FIG. 1. In some examples, the predictive analysis platform 1102 may be the same platform as environment analysis platform 602. In some instances, the environment depicted in FIG. 9 may be a different environment from the environment described with respect to FIG. 1, and/or the predictive analysis platform 1102 may be a different platform from environment analysis platform 602. Referring to FIG. 9, environment information network 900 may include one or more measurement information sources. For example, environment information network 900 may include one or more sensors configured to gather environmental or particle information. The one or more sensors may be deployed in a geographical region. For example, one or more sensors, such as first sensor 901A, may be deployed in urban region 902 (e.g., a city, town, industrial district, and/or other urban regions). In some examples, the one or particle sensors may be deployed to one or more different geographical regions. For example, a first sensor 901A might be deployed in urban region 902, while one or more particle sensors such as second sensor 901B may be deployed in residential region 903 (e.g., a neighborhood, residential district, apartment complex, and/or other residential regions). Additionally or alternatively, in some instances, one or more particle sensors such as third sensor 901C may be deployed in rural region 904 (e.g., a farm, preservation, forest, road, and/or other rural regions).

In some examples, the one or more measurement information sources may be communicatively coupled (e.g., via a wireless data connection) with a computing platform (e.g., for performing predictive analysis). For example, first sensor 901A, second sensor 901B, third sensor 901C, and/or other measurement information sources may be communicatively coupled to predictive analysis platform 1102. Each of first sensor 901A, second sensor 901B, and third sensor 901C may represent multiple sensors. Each of the first sensor 901A, second sensor 901B, and third sensor 901C may be or include a particle sensor, a network of sensors located at different geographical locations within environment 900, a repository of information gathered by multiple unassociated sensors, or sensors included in satellites gathering environmental information. Each of the first sensor 901A, second sensor 901B, and third sensor 901C may be or include a sensor having the components and features of sensor 301 and/or sensor 401 as described herein. Each of the first sensor 901A, second sensor 901B, and third sensor 901C may be supplemented, in providing measurement information, by measurement information stored in a repository of automotive traffic information, in one or more devices gathering environment information of an interior of a building, in one or more devices gathering environment information from a moving vehicle, in one or more anemometers, or in a database corresponding to an enterprise organization. In some examples, the computing platform may receive measurement information (e.g., periodically, continuously, near continuously) from each of the measurement information sources. For example, predictive analysis platform 1102 may continuously or near-continuously receive measurement information (e.g., in real time) from each of first sensor 901A, second sensor 901B, and third sensor 901C. In some instances, the predictive analysis platform 1102 may receive measurement information via real-time streaming from sensor networks alongside daily batch loads from government reference monitors (e.g., US Environmental Protection Agency (EPA) monitors, UK Automatic Urban and Rural Network (AURN) monitors, satellites, and/or other government reference monitors). In other instances, the computing platform may only receive measurement information from a measurement information source at times the measurement information source is considered “active” (e.g., when the measurement information source is powered on, when the measurement information source is actively gathering measurement information, when the measurement information source is actively transmitting measurement information, and/or other times the measurement information source may be considered “active”).

In some instances, as part of or during the receiving measurement information from the first sensor 901A, the second sensor 901B, the third sensor 901C, and/or other measurement information sources described herein, the predictive analysis platform 1102 may apply a sensor correction pipeline (e.g., a method for correcting errors in one or more sensors) to raw sensor values received from the first sensor 901A, second sensor 901B, third sensor 901C, and/or the other measurement information sources. The sensor correction pipeline may included applying tiered correction logic (e.g., a series of computer-executable steps for identifying and remedying errors in a sensor) to normalize data quality across sensor types and manufacturers. The predictive analysis platform 1102 may additionally perform automated drift calibration correction to identify sensors (and/or other devices, such as HVAC or other equipment that contributes to the values detected by the sensors) that are drifting out of calibration and adjust the measurement information or maintenance cycles accordingly. In some instances, as described herein, a measurement information source may be a sensor included in a wearable device. In these instances, as a wearable-wearing individual moves through an environment, the wearable device may continuously collect ground-truth (e.g., direct, localized measurements) personal exposure readings at breathing-zone level across many locations. The predictive analysis platform 1102 may use this high-mobility, high-resolution data stream to validate, correct, and improve the accuracy of a fixed sensor network (e.g., a network comprising first sensor 901A, second sensor 901B, and/or third sensor 901C). The predictive analysis platform 1102 may identify fixed sensors that are drifting out of calibration, detect micro-environment gaps where fixed coverage is insufficient, and provide a spatial model (e.g., the predictive analysis model described herein and/or a spatial model included in the predictive analysis model as described herein) with real-world traversal data from the wearable devices. The combination of wearable devices with a fixed network of sensors provides a distributed calibration instrument (e.g., by comparing the readings/measurements of the wearable devices to the readings/measurements of the fixed network) for the fixed network, improving the accuracy of measurement information from the fixed network (e.g., a network of first sensor 901A, second sensor 901B, third sensor 901C, and/or other sensors) by improving the efficiency and accuracy with which the sensors of the fixed network are calibrated.

In some instances, one or more of the measurement information sources may be or include a wearable device. For example, each of first sensor 901A, second sensor 901B, and third sensor 901C may be a sensor integrated into a wearable device of an individual. The wearable device may be or include a band, smart watch, smart clothing, smart bracelet, fitness tracker, smart belt, and/or other wearable devices. The wearable device may include integrated sensors for detecting one or more measured variables corresponding to one or more attributes of a known particle, one or more additional environment variables, and/or anonymized biometric information of a wearer of the wearable device. The wearable device may be configured to measure particulate matter, heavy metals (e.g., chromium, cadmium, lead, mercury, beryllium, and/or other heavy metals), chemicals, gases (e.g., benzene, ozone, radon, and/or other gases), and per- and polyfluoroalkyl substances (PFAS) in real time and/or continuously or near-continuously. The wearable device may determine exposure concentrations over time for an individual wearing the wearable device and/or for a community using one or more wearable devices. In some instances, the wearable device may capture both air quality exposure and biometric data (e.g., heart rate, blood oxygen saturation (SpO₂), respiratory rate) simultaneously or near-simultaneously and transmit the information to the predictive analysis platform 1102, enabling the predictive analysis platform 1102 to correlate real-time pollution levels with immediate physiological stress responses (e.g., physiological responses corresponding to specific readings/measurements in the biometric data) at the individual level. Over time, the predictive analysis platform 1102 may build a personal sensitivity profile identifying the specific pollutants, concentrations, and durations that trigger a measurable response in that individual, which may differ significantly from population averages. In some examples, the sensitivity profile for an individual may be included in a predictive analysis output to a user (e.g., via a user device) as described herein. In some examples, the wearable device may capture measurement information corresponding to the environment surrounding the wearable device and transmit the measurement information to the predictive analysis platform 1102. In some instances, the wearable device may provide a risk score, as described further herein, based on the captured measurement information. The measurement information gathered by the wearable device and/or other measurement information sources may be anonymized to remove personally identifiable information (PII) before transmitting the measurement information to the predictive analysis platform 1102.

In some instances, the predictive analysis platform 1102 may perform indoor source and behavior fingerprinting through cross-correlation of carbon dioxide (CO₂), formaldehyde, volatile organic compound (VOC), particulate sensor readings, and/or other variables. The indoor source and behavior fingerprinting may classify outdoor and indoor pollution events by type and source, including differentiation of occupancy-driven activity from temperature-driven chemical offgassing (e.g., the releasee of accumulated VOCs and other chemicals) from building materials and furnishings. In some examples, the predictive analysis platform 1102 may perform the indoor source and behavior fingerprinting by identifying known attributes of the CO₂, formaldehyde, VOC, particulate sensor readings, and/or other variables using the techniques described herein with respect to the environmental analysis model (e.g., as described herein with respect to FIG. 7). The predictive analysis platform 1102 may include the indoor source and behavior fingerprinting in the predictive analysis described herein.

The measurement information gathered by the one or more measurement information sources and received by the computing platform may include one or more measured variables corresponding to one or more attributes of a known particle, and/or one or more additional environment variables. The one or more additional environment variables may include one or more of: wind speed, wind direction, particle concentrations, gas concentrations, temperature, relative humidity, air pressure, dew point, vibration frequency, noise level, ventilation status, altitude, topographical information, occupancy, traffic count, mold growth information, mildew growth information, population information, luminosity information, precipitation information, cloud ceiling height, audio measurements, or virus detection information. In some instances, a single measurement information source (e.g., first sensor 901A, a wearable device, and/or other measurement information sources) may gather a set of measurement information. For example, first sensor 901A may gather both a measured particle count and a measured particle size. In some instances, measurement information may additionally or alternatively come from a survey through an application or website on a mobile device (e.g., user device 1108, as described herein).

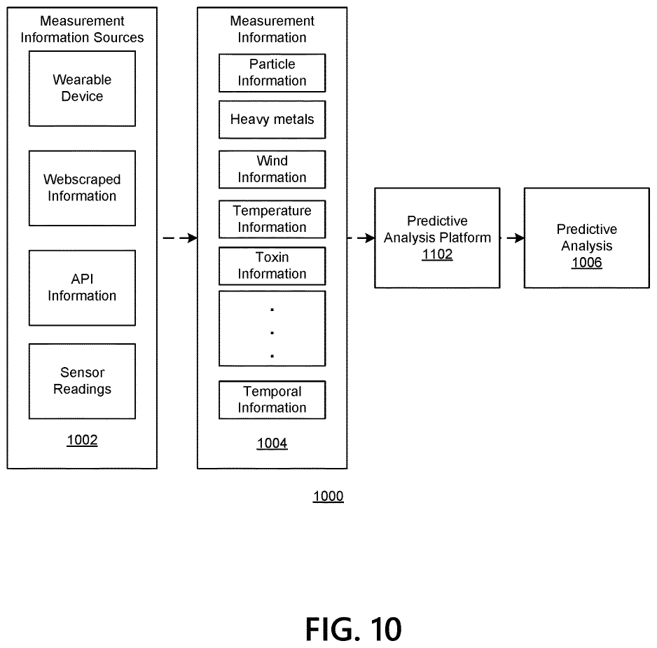

FIG. 10 depicts an illustrative event flow for predictive analysis using measurement information sources and measurement information. In some instances, the event flow depicted in FIG. 10 may include some or all of the same information and components as the event flow in FIG. 2A. In some examples, the predictive analysis platform 1102 may be the same platform as environment analysis platform 602. In some instances, the predictive analysis platform 1102 may be a different platform from environment analysis platform 602. Referring to FIG. 10, one or more measurement information sources 1002 may be configured to gather measurement information 1004 corresponding to a particular environment and/or individual and send the measurement information to the predictive analysis platform 1102. The one or more measurement information sources 1002 may be or include one or more of: a wearable device having integrated sensors, a particle sensor (e.g., a sensor such as first sensor 901A, second sensor 901B, third sensor 901C, sensor 301, and/or sensor 401 as described herein), a network of particle sensors located at different geographical locations, a repository of information gathered by multiple unassociated sensors, satellites gathering environmental information, a repository of automotive traffic information (e.g., a repository of information gathered by one or more of cameras, radar sensors, lidar sensors, road cables, and/or other sources of traffic information), one or more particle sensors gathering measurement information 1004 of the interior of a building, one or more devices gathering measurement information 1004 from a moving vehicle, one or more anemometers, or a database corresponding to an enterprise organization. In some examples, the one or more measurement information sources 1002 may be configured to send the measurement information 1004 directly to the predictive analysis platform 1102. Additionally or alternatively, in some instances the one or more measurement information sources 1002 may be configured to indirectly send the measurement information 1004 to the predictive analysis platform 1102. For example, the one or more measurement information sources 1002 may send the measurement information 1004 to a repository (e.g., a web-based server, database, cloud storage system, and/or other repositories). In these examples, the predictive analysis platform 1102 may retrieve the measurement information from the repository (e.g., via webscraping methods, application programming interface (API) calls, information transfer requests, and/or other methods).

The measurement information sources 1002 may gather the measurement information 1004 corresponding to the particular environment and/or individual. The measurement information sources 1002 may gather the measurement information 1004 by using cameras, lasers, filtration devices, anemometers, integrated sensors in wearable devices (e.g., accelerometers, gyroscopes, photoplethysmography trackers, or the like) , and/or other methods. The measurement information 1004 gathered by the one or more measurement information sources 1002 and received by the predictive analysis platform 1102 may include some or all of the environment information described with respect to FIG. 2A. In this way, the measurement information 1004 utilized for predictive analysis may combine the environment information as described herein with additional health parameters gathered by other sensors or wearable devices. The measurement information 1004 may also include temporal information (e.g., the time of day, the current month, the current year, a range of time during which certain health parameters were present, or the like). The measurement information 1004 may include one or more of: particulate matter measurements, chemical measurements, heavy metal measurements (e.g., chromium, cadmium, lead, mercury, beryllium, and/or other heavy metals), gas measurements (e.g., benzene, ozone, radon, and/or other gases), environmental toxin measurements, per- and polyfluoroalkyl substance (PFAS) measurements, biometric information of a wearer of a wearable device (e.g., heart rate, oxygen level, blood oxygen saturation, skin temperature, sleep patterns, heart rate variability, blood pressure), audio measurements, wind speed, wind direction, particle concentration, gas concentration, temperature, relative humidity, air pressure, dew point, vibration frequency, noise level, ventilation status, altitude, topographical information, occupancy, traffic counts, mold growth information, mildew growth information, population information, luminosity information, precipitation information, cloud ceiling height, or virus detection information.

In some instances, the predictive analysis platform 1102 may perform particulate fingerprinting (e.g., identification of particulates based on their attributes, as described herein) to characterize particulate matter, gases, and/or other organic matter by composition, size distribution, and/or source signature as described herein. The particulate fingerprinting may use machine learning to identify and attribute pollution to specific source types, such as traffic, industrial, agricultural, and/or indoor sources. For example, the particulate fingerprinting may be performed using the environmental analysis model and as described herein with respect to steps 702-718 and FIG. 7. The particulate fingerprinting may be included in the predictive analysis generated by the predictive analysis platform 1102 as described further herein.

Additionally or alternatively, the measurement information 1004 may include information corresponding to a healthcare service provider. The information corresponding to the healthcare service provider may include one or more of: therapeutic information for treating diseases, pathology information, community disease information, infection rates for a disease or virus, or infection vectors for a disease or virus. In some instances, the measurement information 1004 may include information from blinded electronic medical records and medical claims identifying where respiratory illnesses, cancers, cardiovascular diseases, and/or blood disorders are being identified geographically by locality. The measurement information 1004 may additionally or alternatively include information from a pathology lab, transmitted to the predictive analysis platform 102 via a communication interface from a computing device in, or associated with, the pathology lab.

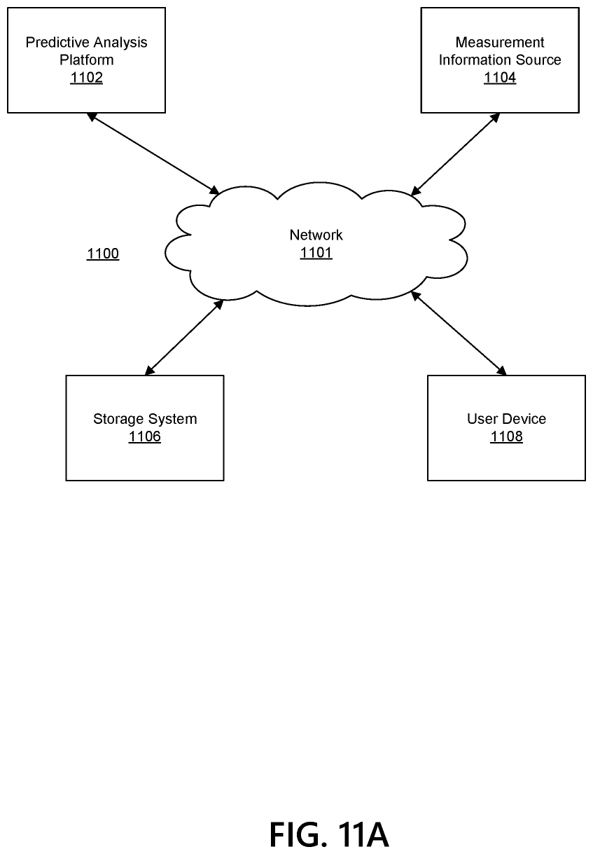

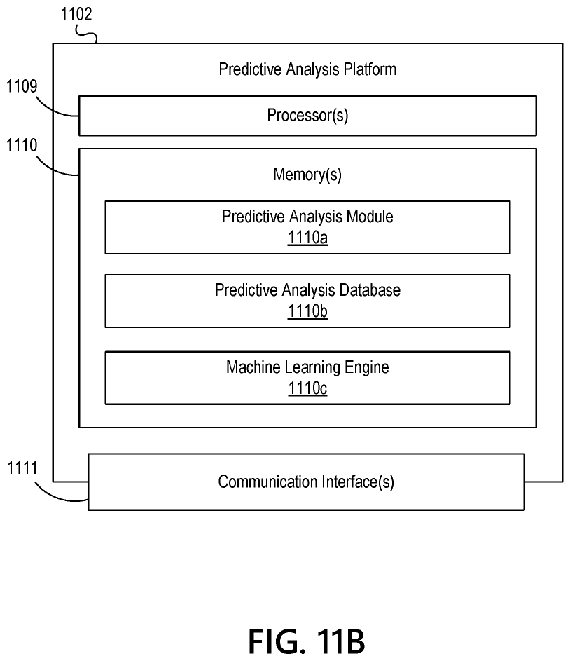

The predictive analysis platform 1102 may receive the measurement information 1004 from the measurement information sources 1002 via one or more wired or wireless data connections which may, e.g., be established via a communication interface communicatively coupled to the predictive analysis platform 1102 (e.g., communication interface 1111, as illustrated in FIG. 11B, and/or other communication interfaces).

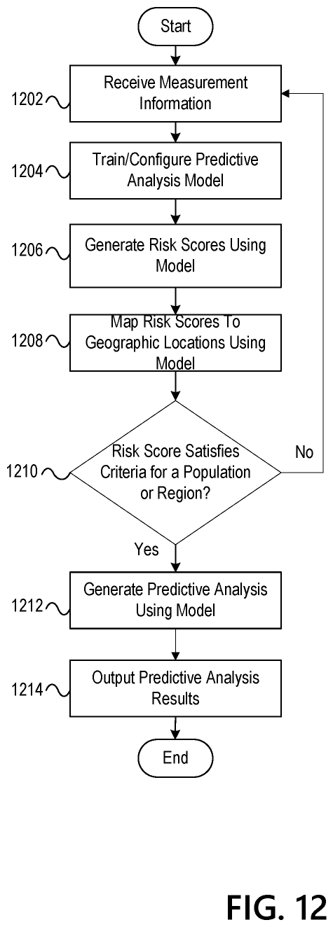

The predictive analysis platform 1102 may generate a predictive analysis 1006 based on the measurement information 1004. For example, the predictive analysis platform 1102 may generate one or more risk scores indicating a likelihood of the presence of a health risk at a geographic location using a predictive analysis model. The predictive analysis platform 1102 may generate a mapping comprising the one or more risk scores and the geographic location. The predictive analysis platform 1102 may generate, using the predictive analysis model and based on the mapping and the one or more risk scores, the predictive analysis 1006. The predictive analysis 1006 may include an explanation of the one or more risk scores and an indication of the health risk at the geographic location. The predictive analysis 1006 may identify an individual, population, or region's risk of developing respiratory diseases, cardiovascular diseases, cancers, and/or blood disorders based on the level of exposure to varying environmental toxins. Creation of the predictive analysis model may employ machine learning as illustrated in FIG. 12 and described further herein.

In some instances, the predictive analysis platform 1102 may perform automated anomaly detection using combined statistical and machine learning models to identify pollutant spikes, hot spots (e.g., a bounded geographic region having a reading or measurement for a health parameter above a threshold), patterns, and/or sensor anomalies across the measurement information sources. For example, the predictive analysis platform 1102 may utilize a statistical model (e.g., a statistical regression model, or the like) combined with a machine learning model (e.g., the predictive analysis model, as described herein) to compare machine-generated data with statistical data to identify the spikes, hot spots, patterns, and/or sensor anomalies across the measurement information sources. The predictive analysis platform 1102 may additionally perform spatial pattern analysis, including geographic clustering and gradient mapping across census tracts, local super output areas (LSOAs), and/or other geographic boundaries. The predictive analysis platform 1102 may perform the spatial pattern analysis using the predictive analysis model, as described herein. The predictive analysis platform 1102 may further perform temporal pattern analysis, including hourly and daily heatmaps and seasonal-trend decomposition (e.g., deconstructing a time series of measurement information into several components, each representing an underlying trend, season, or residual) to surface cyclical pollution patterns. The predictive analysis platform 1102 may perform the temporal pattern analysis using the predictive analysis model, as described herein. In some examples, the predictive analysis platform 1102 may additionally perform sensor network clustering. For example, the predictive analysis platform 1102 may, using a predictive analysis model as described herein, identify groupings and correlated behaviors across distributed sensor networks (e.g., by mapping patterns between different sensors in the distributed sensor network, such as a network comprising first sensor 901A, second sensor 901B, and/or third sensor 901C).

In some instances, the predictive analysis platform 1102 may perform building envelope infiltration modeling using the live relationship between simultaneous indoor and outdoor sensor readings (e.g., from one or more indoor measurement information sources and one or more outdoor measurement information sources as described herein). The building envelope infiltration modeling may quantify how effectively a building's envelope (e.g., walls, windows, ventilation) filters or amplifies outdoor pollution events. The predictive analysis platform 1102 may generate a per-building infiltration coefficient that updates dynamically. Combined with outdoor forecasts, the building envelope infiltration modeling may enable predictive indoor exposure alerts before an outdoor event reaches harmful indoor levels, providing occupants or building managers actionable lead time. For example, the predictive analysis platform 1102 may generate the per-building infiltration coefficient and output, with or as part of the predictive analysis, the predictive indoor exposure alert based on the predictive analysis.

FIGS. 11A-11B depict illustrative computing systems for predictive analysis in accordance with one or more example embodiments. In some instances, the computing systems depicted in FIGS. 11A-11B may be the same as the computing systems depicted in FIGS. 6A-6B. In some examples, the predictive analysis platform 1102 may be the same platform as environment analysis platform 602. In some instances, the computing systems depicted in FIGS. 11A-11B may be different from the computing systems depicted in FIGS. 6A-6B, and/or the predictive analysis platform 1102 may be a different platform from environment analysis platform 602. Referring to FIG. 11A, system 1100 may include one or more computer systems. For example, system 1100 may include the predictive analysis platform 1102, a measurement information source 1104, a storage system 1106, and a user device 1108.

Predictive analysis platform 1102 may be a computer system that includes one or more computing devices (e.g., servers, server blades, or the like) and/or other computer components (e.g., processors, memories, communication interfaces) that may be configured to operate, train and/or execute one or more machine learning models, such as predictive analysis models. For example, the predictive analysis platform 1102 may train the one or more predictive analysis models to generate risk scores and predictive analyses for one or more environments, individuals, and/or populations. In other configurations, the predictive analysis platform 1102 may implement a generative artificial intelligence model (e.g., using prompt engineering), such as a generative large language model (LLM). In some instances, predictive analysis platform 1102 may be controlled or otherwise maintained by an enterprise organization such as a healthcare organization, insurance provider, or environmental analysis organization. Although shown as an independent predictive analysis platform 1102, in some instances, the predictive analysis platform 1102 may be part of and/or otherwise integrated into the user device 1108 without departing from the scope of the disclosure.

Measurement information source 1104 may be and/or otherwise include one or more computing devices (e.g., servers, server blades, and/or other devices) and/or other computer components (e.g., processors, memories, communication interfaces) that may be configured to gather measurement information corresponding to a particular environment, individual, and/or population and send the measurement information to the predictive analysis platform 1102. Although shown as an independent information source, in some instances, the measurement information source 1104 may be or include one or more information sources, such as measurement information sources 1002 as described herein, and/or additional wearable devices. The measurement information source 1104 may be or include one or more of: a particle sensor (e.g., a sensor such as sensors 901A-901C, sensor 301, and/or sensor 401), a network of sensors located at different geographical locations, a repository of information gathered by multiple unassociated sensors, satellites gathering environmental information, a repository of automotive traffic information, one or more devices gathering environment information of the interior of a building, one or more devices gathering environment information from a moving vehicle, one or more anemometers, a database corresponding to an enterprise organization, and/or wearable devices having integrated sensors.

Storage system 1106 may be and/or otherwise include one or more computing devices (e.g., servers, server blades, and/or other devices) and/or other computer components (e.g., processors, memories, communication interfaces) that may be configured to create, host, modify, and/or otherwise validate a storage system. In some instances, the storage system 1106 may be a cloud storage system (e.g., a distributed ledger). In some instances, the storage system 1106 may be other types of storage systems, such as local storage, on-premises servers, hybrid storage systems, and/or other storage configurations. The storage system 1106 may be synchronized across multiple nodes (e.g., sites, institutions, geographical locations, and/or other nodes) and may be accessible by multiple users (who may, e.g., be employees or clients of an enterprise organization). The data stored at the databases of storage system 1106 may include any of the data (e.g., measurement information, health information, risk scores, predictive analyses, and/or other information) stored at and/or created by predictive analysis platform 1102 and/or any additional data. In some instances, the databases stored on storage system 1106 may be accessed by, validated by, and/or modified by any of, user device 1108, predictive analysis platform 1102, and/or other devices.

User device 1108 may be a computing device (e.g., laptop computer, desktop computer, mobile device, tablet, smartphone, server, server blade, and/or other device) and/or other data storing or computing component (e.g., processors, memories, communication interfaces, databases) that may be configured to transfer data between users and/or perform other user functions (e.g., receiving display commands, and/or other functions). In one or more instances, user device 1108 may correspond to a user account (which may, e.g., be associated with an employee of an enterprise organization, may be a client of the enterprise organization, may be otherwise associated with the enterprise organization, or the like). In some instances, user device 1108 may be a device corresponding to a healthcare service provider, a health care insurance provider, a regulatory agency, a pharmaceutical company, or any other entity associated with providing health services. In one or more instances, the user device 1108 may be configured to communicate with one or more systems (e.g., predictive analysis platform 1102, measurement information source 1104, and/or other systems) to perform a data transfer, receive display commands, and/or to perform other functions. Providing predictive analysis information to the user device 1108 may equip consumers, health insurers, pharmaceutical companies, and/or other entities to understand the risk of where individuals live, who they cover, and what medicines may have the greatest beneficial impact.

System 1100 also may include one or more networks, which may interconnect predictive analysis platform 1102, measurement information source 1104, storage system 1106, user device 1108. For example, system 1100 may include a network 1101 (which may interconnect, e.g., predictive analysis platform 1102, measurement information source 1104, storage system 1106, and user device 1108).

In one or more arrangements, predictive analysis platform 1102, measurement information source 1104, storage system 1106, user device 1108 may be a wearable device (e.g., the wearable device that collected a portion of the measurement information 1004), a laptop, a mobile phone, or any type of computing device capable of sending and/or receiving requests and processing the requests accordingly. For example, predictive analysis platform 1102, measurement information source 1104, storage system 1106, user device 1108, and/or the other systems included in system 1100 may, in some instances, be and/or include server computers, desktop computers, laptop computers, tablet computers, smart phones, or the like that may include one or more processors, memories, communication interfaces, storage devices, and/or other components. As noted above, and as illustrated in greater detail below, any and/or all of predictive analysis platform 1102, measurement information source 1104, storage system 1106, user device 1108 may, in some instances, be special-purpose computing devices configured to perform specific functions. For example, each may include memory that includes computer readable instructions that, when executed by a processor, cause the special-purpose computing device to perform one or more functions described herein.

Referring to FIG. 11B, predictive analysis platform 1102 may include one or more processors 1109, memory 1110, and communication interface 1111. A data bus may interconnect processor 1109, memory 1110, and communication interface 1111. Communication interface 1111 may be a network interface configured to support communication between predictive analysis platform 1102 and one or more networks (e.g., network 1101, or the like). Communication interface 1111 may be communicatively coupled to the processor 1109. Communication interface 1111 may be configured to receive sets of measurement information from one or more measurement information sources via one or more wireless data connections. Memory 1110 may include one or more program modules having instructions that, when executed by processor 1109, cause predictive analysis platform 1102 to perform one or more functions described herein and/or one or more databases that may store and/or otherwise maintain information which may be used by such program modules and/or processor 1109. In some instances, the one or more program modules and/or databases may be stored by and/or maintained in different memory units of predictive analysis platform 1102 and/or by different computing devices that may form and/or otherwise make up predictive analysis platform 1102. For example, memory 1110 may have, host, store, and/or include predictive analysis module 1110a, predictive analysis database 1110b, and machine learning engine 1110c.

Predictive analysis module 1110a may have instructions that direct and/or cause predictive analysis platform 1102 to perform predictive analysis (e.g., as described with respect to FIGS. 10 and 12). Predictive analysis database 1110b may have instructions causing predictive analysis platform 1102 to store measurement information, health information, risk scores, and/or predictive analyses (that may, e.g., be used to perform predictive analysis). Machine learning engine 1110c may contain or include instructions causing predictive analysis platform 1102 to train, implement, and/or update a predictive analysis model (e.g., as described with respect to FIG. 12). In some instances, machine learning engine 1110c may be used by predictive analysis platform 1102 and/or predictive analysis module 1110a to refine and/or otherwise update methods for predictive analysis, and/or other methods described herein.

FIG. 12 depicts an illustrative method for predictive analysis using a predictive analysis model in accordance with one or more example embodiments. Referring to FIG. 12, at step 1202, the predictive analysis platform 1102 may receive, via a communication interface from one or more measurement information sources, a set of measurement information. The set of measurement information may correspond to health parameters (e.g., conditions that indicate a health risk or information that is neutral towards, or counteracts, a health risk) at a geographic location. In some instances, the set of measurement information may be anonymized to remove personally identifiable information (PII) before processing. The set of measurement information may include information gathered by a wearable device, information gathered by one or more environmental sensors, and/or information corresponding to a healthcare service provider. The information gathered by the wearable device may include one or more of: particulate matter measurements, chemical measurements, heavy metal measurements, gas measurements, environmental toxin measurements, PFAS measurements, biometric information of the wearer of the wearable device, audio measurements, wind speed, wind direction, particle concentration, gas concentration, temperature, relative humidity, air pressure, dew point, vibration frequency, noise level, ventilation status, altitude, topographical information, occupancy, traffic counts, mold growth information, mildew growth information, population information, luminosity information, precipitation information, bacteria detection information, or virus detection information. The information gathered by the one or more environmental sensors may include one or more of: particulate matter measurements, chemical measurements, heavy metal measurements, gas measurements, environmental toxin measurements, PFAS measurements, audio measurements, wind speed, wind direction, particle concentration, gas concentration, temperature, relative humidity, air pressure, dew point, vibration frequency, noise level, ventilation status, altitude, topographical information, occupancy, traffic counts, mold growth information, mildew growth information, population information, luminosity information, precipitation information, bacteria detection information, or virus detection information. The information corresponding to the healthcare service provider may include one or more of: therapeutic information for treating diseases, pathology information, community disease information, infection rates for a disease or virus, or infection vectors for a disease or virus. In some instances, the set of measurement information may include information from blinded electronic medical records and medical claims identifying where respiratory illnesses, cancers, cardiovascular diseases, and/or blood disorders are being identified geographically by locality. Additionally or alternatively, the set of measurement information may include information from a pathology lab.

At step 1204, the predictive analysis platform 1102 may train/configure the predictive analysis model. In training the predictive analysis model, the predictive analysis platform 1102 may configure and/or otherwise train the predictive analysis model to generate risk scores and predictive analyses based on measurement information. The predictive analysis platform 1102 may train, based on historical measurement information, the predictive analysis model. The historical measurement information may be or include measurement information from any of the measurement information sources described herein and corresponding to a prior health risk. For example, the historical measurement information may be or include measurement information, previously received by the predictive analysis platform 1102 or received with the measurement information in step 1202, indicating conditions in a geographic location during an occurrence of a prior health risk (e.g., a disease or viral outbreak, a hazardous weather event, a toxic spill, or the like). In some instances, the predictive analysis platform 1102 may use logistic regression to establish causal and/or associated relationships between environmental hazards and health outcomes, which may form the basis of the risk scores. In some instances, the predictive analysis platform 1102 may apply weather-deweathering techniques via bootstrap resampling with the predictive analysis model to statistically isolate human-caused pollution signals from meteorological confounders. The weather-deweathering may include repeated bootstrap resampling of weather variables to produce weather-normalized concentration trends that distinguish policy or emissions-driven changes from natural variability. In this way, the predictive analysis platform 1102 may train/configure the predictive analysis model to produce predictive analyses that accurately indicate whether a risk score corresponds to a human-caused health risk or a natural/meteorological health risk. In some examples, the predictive analysis platform 1102 may also or alternatively train the predictive analysis model using metadata. For example, metadata comprising information that correlates data across sets (e.g., that correlates the historical measurement information to current measurement information) may be used to train the model. In these examples, the metadata correlating data across sets may be used by the predictive analysis platform 1102 to cause the predictive analysis model to store one or more correlations between data (e.g., data collected in the same time interval, data collected by the same device (e.g., a wearable device as described herein), or multiple samples of the same data). The stored correlations may be used by the predictive model to identify similarities, patterns, or the like between measurement information (e.g., measurement information 1004) received by the predictive analysis platform 1102 and historical measurement information in future iterations of the method described in FIG. 12, in order to generate risk scores, mappings, and/or predictive analyses as described herein. Training the predictive analysis model may configure the predictive analysis model to generate the one or more risk scores by comparing the set of measurement information to historical information indicating a given health risk, generate a mapping, and generate, based on the mapping and the one or more risk scores, a predictive analysis.

In some examples, the predictive analysis model may be a deterministic or probabilistic model trained based on training data (e.g., the historical measurement information described above). In these examples, the model may be configured/trained by the predictive analysis platform 1102 causing the model to process the training data using one or more machine learning techniques. For example, the predictive analysis platform 1102 may cause the predictive analysis model to process the training data by applying natural language processing, natural language understanding, supervised machine learning techniques (e.g., regression, classification, neural networks, support vector machines, random forest models, naïve Bayesian models, supervised fine-tuning (SFT), reinforcement learning from human feedback (RLHF) or other supervised techniques), unsupervised machine learning techniques (e.g., principal component analysis, hierarchical clustering, K-means clustering, or other unsupervised techniques), or other techniques. In some examples, the predictive analysis model may have previously processed the training data using one or more of the above techniques (e.g., prior to the predictive analysis platform 1102 implementing or otherwise configuring the predictive analysis model). For example, the predictive analysis model may be a pre-trained commercial or bespoke model implemented by the predictive analysis platform 1102

In some instances, the predictive analysis model may be a generative artificial intelligence model. For example, the predictive analysis model may be a large language model, a metadata model having different modalities (e.g., text, image, video, audio, or the like), a generative pre-trained transformer, a generative adversarial network, a variational autoencoder, or any other form of generative artificial intelligence (GenAI) model. In one or more of these examples, the training data may include a volume of information sufficient to train a large language model, which may exceed the amount of information that other machine learning models are capable of processing. Thus, it may be beneficial to use a large language model over other machine learning models when processing this large volume of information. In some examples, the predictive analysis platform 1102 may configure the predictive analysis model by implementing, activating, or otherwise using, as the predictive analysis model, a commercial off-the-shelf GenAI model that was previously trained, as described above, based on the training data. The data may include correlations, indications, or the like of what constitutes measurement information for a particular health risk. For example, the training data may include techniques, templates, instructions, or the like for generating risk scores, mappings, and/or predictive analyses that have previously been identified (e.g., by another model, an algorithm, or by a manual process) as important or useful in identifying whether a health risk is present at a geographic location.

The use of a generative artificial intelligence model may allow for a more flexible model that can continuously be updated with new inputs from wearable sensors that may have a direct application programming interface (API) connection for more seamless data evaluation. In some examples, the predictive analysis platform 1102 may utilize a look-back period of 5-10 years where historical data is available from public sources and/or other data repositories. In these instances, the predictive analysis platform 1102 may generate the one or more risk scores by providing a prompt to the predictive analysis model, the prompt comprising the set of measurement information and one or more instructions for the predictive analysis model to generate the one or more risk scores based on historical information indicating a given health risk.

In some examples, the predictive analysis model may be the environmental analysis model described herein. For example, the predictive analysis platform 1102 may train/configure the environmental analysis model (already trained and used to perform the functions described herein with respect to FIG. 7) to perform the additional functions described herein as being performed by the predictive analysis model. In some examples, the predictive analysis model and the environmental analysis model may be different models.

At step 1206, the predictive analysis platform 1102 may generate, using the predictive analysis model and the set of measurement information, one or more risk scores indicating a likelihood of the presence of a health risk at the geographic location. In generating the one or more risk scores, the predictive analysis platform 1102 may provide the set of measurement information to the predictive analysis model to cause the predictive analysis model to determine, based on applying weighted values to portions of the set of measurement information, a likelihood of the presence of the health risk. For example, the predictive analysis platform 1102 may provide instructions or a prompt to the predictive analysis model directing the predictive analysis model to weight certain types of measurement information (i.e., portions of the set of measurement information) higher than other types (e.g., a high wind speed may be given a lower weight than an indication of the presence of an airborne toxin). The predictive analysis platform may use algorithms or formulas to calculate, based on the totality of the measurement information, and appropriate risk score for a given health risk. In some examples, the predictive analysis platform may generate multiple risk scores based on the same measurement information, with each risk score corresponding to a different health risk. The weights applied to portions of the set of measurement information may be predetermined (e.g., by a user of the predictive analysis platform 1102) and stored in memory (e.g., memory 1110).

The predictive analysis platform 1102 may generate, by converting the likelihood to a numerical value, a risk score for at least one of: an individual, a population, and an environment corresponding to the geographic location. The one or more risk scores may indicate a health risk such as an individual, population, or region's risk of developing respiratory diseases, cardiovascular diseases, cancers, and/or blood disorders based on the level of exposure to varying environmental toxins. In some examples, the one or more risk scores may indicate, based on measurement information such as measure variables corresponding to attributes of a known particle or additional environment variables as described herein, a likelihood of a natural disaster or other types of physical health risks. In some instances, the predictive analysis platform 1102 may update the sensitivity and specificity of the data over time as additional data points are collected. Risk scores may be created independently per disease or event and then rolled up into a composite score which may allow for the use of one value to make cross comparisons between individuals and collective populations. In some instances, the predictive analysis platform 1102 may apply various statistical or machine-learning models to generate the risk scores. For example, the risk scores may be generated using the predictive analysis model described herein (e.g., by prompting the predictive analysis to generate the risk scores using the measurement information). In some examples, the predictive analysis platform 1102 may use a statistical analysis technique additionally or alternatively to the predictive analysis model. For example, the predictive analysis platform 1102 may use a land-use regression (LUR) analysis to describe spatial variation in air pollution and estimated concentrations at locations without sensors, then use the spatial variations and estimated concentrations to increase or decrease the risk score. For example, if the spatial variation or estimated concentrations exceed a predetermined threshold for spatial variation or estimated concentration, the predictive analysis platform 1102 may increase the risk score, but if the amount of pollution does not meet or exceed the predetermined threshold for spatial variation or estimated concentration, the predictive analysis platform 1102 may decrease the risk score. The LUR analysis may use linear regression to relate measured pollution at specific locations with geospatial data such as traffic intensity, population density, road length, land cover, road proximity, and/or farm proximity. The predictive analysis platform 1102 may additionally or alternatively apply an exposure model to calculate the amount of a pollutant a person or population receives by integrating LUR-modeled concentrations with human behavior data. The exposure model may include simple models focusing on long-term ambient exposure based on address information, and/or complex dynamic models using GPS tracking to calculate short-term personal exposure during daily activities. Based on the calculation of the amount of pollutant a person or population receives, the predictive analysis platform 1102 may increase or decrease the risk score. For example, if the amount of pollution a given person or population receives in a given timeframe exceeds a predetermined threshold for pollutant exposure, the predictive analysis platform 1102 may increase the risk score, but if the amount of pollution does not meet or exceed the predetermined threshold for pollutant exposure, the predictive analysis platform 1102 may decrease the risk score.

At step 1208, the predictive analysis platform 1102 may generate a mapping comprising the one or more risk scores and the geographic location. In generating the mapping, the predictive analysis platform 1102 may map the risk scores and/or the measurement information to specific geographic locations. The mapping may include a geospatial assessment by geographic region (e.g., by zipcode, county, city, and/or other geographic regions) to create a heat map of environmental exposures and/or health risks. In some instances, the geospatial assessment may be performed cross-sectionally to enable comparison of environmental exposures and health outcomes across different geographic regions at a given point in time. In some examples, the predictive analysis platform 1102 may generate the mapping using the predictive analysis model. For example, the predictive analysis platform 1102 may include, in the original prompt or instructions for the predictive analysis model in step 1206 or in an additional prompt or instructions, commands for the predictive analysis model to generate a mapping as described above

At step 1210, the predictive analysis platform 1102 may determine whether or not the one or more risk scores satisfy a criteria for a population or region. The criteria may be or include a threshold value, a threshold range, a limit on the risk score, and/or other criteria. In some instances, the predictive analysis platform 1102 may determine hot spots for disease diagnosis and medical utilization over time, including physician visits, related surgical procedures, and pharmaceutical drug use for related respiratory illnesses, cardiovascular diseases, cancers, and blood disorders. The predictive analysis platform 1102 may assign a threshold amount of occurrences of these events, at a given hotspot, and use that threshold for the comparison. For example, if fifty physician visits occur within a month in a given geographical region, the threshold that a risks core must satisfy to indicate a health risk may be lowered compared to a scenario where only five physician visits occur within a month in the given geographical region, because the presence of the hot spot may indicate a higher likelihood of a health risk being present. In some instances, the predictive analysis platform 1102 may track the cumulative exposure profile for an individual, community, population, or the like over weeks and/or months, detecting whether their risk is drifting toward or away from clinically significant thresholds. The clinically significant threshold may be determined by a source of the measurement information (e.g., a clinic) and/or by instructions provided to the predictive analysis platform 1102 by a user. The predictive analysis platform 1102 may apply trend analysis to characterize the direction, magnitude, and projected future value of the trajectory of the exposure profile. Threshold proximity scores may compare the current exposure level to defined health risk thresholds at one or more severity levels. Drift alerts may be generated (e.g., as part of the predictive analysis described herein) when the trajectory is directed toward a threshold and the projected concentration at a defined forecast horizon exceeds the threshold, subject to a minimum trend duration requirement to exclude transient effects. In this way, the predictive analysis platform 1102 may provide (e.g., as part of the predictive analysis as described herein) an early-warning capability capable of flagging a region, individual, or population with a combined exposure on an upward trend before a threshold is crossed and produces longitudinal exposure records (e.g., included in the predictive analysis) that can be output to an epidemiological research organization, insurer, disease management organization, or the like. Based on or in response to determining that the one or more risk scores fail to satisfy the criteria, the predictive analysis platform 1102 may return to step 1202 to receive additional measurement information. Based on or in response to determining that the one or more risk scores satisfy the criteria, the predictive analysis platform 1102 may proceed to generate the predictive analysis (e.g., as described below at step 1212). In some examples, the predictive analysis platform 1102 may maintain per-user longitudinal pollutant concentration records comprising the one or more risk scores and stored, for example, in the predictive analysis database 1110b. The per-user longitudinal pollutant concentration records may include pollutant concentration measurements associated with the individual over days, weeks, months, and/or years and may also include an indicator of an association between the pollutant concentration measurements and the one or more risk scores. The drift alerts may be delivered (e.g., during output of the predictive analysis as described below at step 1214) to the user via the user device 1108 and, with permission from the user, to registered clinicians, insurance underwriters, and/or other authorized recipients, enabling early intervention before threshold crossing occurs.

At step 1212, the predictive analysis platform 1102 may generate, using the predictive analysis model and based on the mapping and the one or more risk scores, a predictive analysis. The predictive analysis may include an explanation of the one or more risk scores and an indication of the health risk at the geographic location. The explanation may include information indicating the portions of the set of measurement information that contributed to each of the one or more risk scores. The predictive analysis platform 1102 may generate the explanation using the predictive analysis model. For example, the predictive analysis platform 1102 may provide instructions or a prompt to the predictive analysis model to cause the predictive analysis model to generate an explanation of the one or more risk scores. The explanation may include a description of which portions of the set of measurement information received at step 1202 contributed to each risk score generated by the predictive analysis platform 1102 as well as the weights applied to those portions of the set of measurement information. The indication of the health risk may be a text summary, score, graphic, or other visual indicator of the type of health risk (e.g., a disease, virus, bacteria, airborne toxin, natural disaster, or other health risks described herein) and a level of risk associated with the health risk (e.g., low, medium, high, severe, etc.) or risk score (e.g., percentage, integer, letter grade, etc.).

The predictive analysis may additionally or alternatively include a summary of the health risk (e.g., a heightened risk of a disease in a county, city, and/or other geographic region). In some instances, the predictive analysis may track the flow and dissipation of pollution, including how long it takes to dissipate harmful chemicals, gases, or the like, and may include a flow map of this dissipation. The predictive analysis may additionally or alternatively include one or more instructions for responding to the health risk (e.g., “interventions”). The one or more instructions for responding to the health risk may include one or more of: a visual representation of geographic areas associated with the health risk (which may, e.g., comprise the mapping generated by the predictive analysis platform 1102), a suggested route for avoiding the health risk, a recommendation for placement of environmental sensors for gathering information of the health risk, an agenda, schedule, or report on event conditions, a text description of the health risk, or an audio description of the health risk.

In some instances, the predictive analysis may include cross-environment personal exposure attribution. The predictive analysis platform 1102 may combine time-activity patterns with location-specific indoor and outdoor air quality measurements across home, workplace, and commute environments, from the measurement information, to disaggregate an individual's total daily pollution dose and attribute it to each environment. The predictive analysis may deliver this attribution as a quantified percentage contribution per environment (e.g., indicating that 65% of an individual's PM 2.5 exposure comes from a morning commute, 25% from the individual's home overnight, and 10% from the individual's workplace). This per-environment breakdown may be used to rank interventions (e.g., the one or more instructions for responding to the health risk) by their expected dose reduction impact. The intervention ranking may include, for example, a ranking of suggested routes for avoiding a health risk, a ranking of recommended locations for placement of environmental sensors, and/or other rankings of intervention options. In some instances, personal exposures may be systematically underestimated for individuals who commute long distances, because such individuals spend substantial time in high-pollution transit environments that are not captured by residential monitoring. Similarly, indoor exposures at both home and workplace may be dominated by distinct source profiles including cooking, candle burning, elevated perfume or scents in the home, cleaning products, building materials, printers, and HVAC systems, which are not represented by outdoor ambient readings. The predictive analysis platform 1102 may use GPS data, dynamic and static data from sensors (e.g., first sensor 901A, second sensor 901B, third sensor 901C), wearable sensor data, and/or individual survey input to calculate the individual's daily, seasonal, and yearly exposure. The predictive analysis platform 1102 may include, in the predictive analysis, an average of the exposure of an individual over these time periods to provide a comprehensive exposure profile that accounts for time spent in each environment

In some instances, the predictive analysis platform 1102 may maintain an intervention library comprising a plurality of candidate interventions (e.g., suggested actions to take for responding to different health risks). For each candidate intervention in the intervention library, the predictive analysis platform 1102 may apply an expected concentration reduction function to a current environment-specific pollutant concentration for a user to track past concentrations, monitor concentrations during the intervention, and project post-intervention concentrations. In some instances, the predictive analysis may include an intervention recommendation providing score-driven prioritization of actionable risk reduction recommendations tailored to a specific risk score for an individual, community, city, population, or the like. The predictive analysis platform 1102 may apply expected concentration reduction functions to the current environment-specific pollutant concentrations for a user (e.g., of the predictive analysis platform 1102 and/or of a wearable device providing measurement information to the predictive analysis platform 1102) associated with the measurement information received at step 1202 to project post-intervention concentrations and compute projected post-intervention risk scores. For example, the predictive analysis platform 1102 may compute the risk score reduction associated with each intervention and rank the interventions by projected risk score impact, as described herein with respect to step 1206. The ranked intervention list may be added to the predictive analysis platform and include quantified projected exposure reductions, risk score changes, and time horizons, enabling evidence-based prioritization of personalized interventions. The predictive analysis platform 1102 may additionally perform intervention monitoring and effectiveness assessment with continuous or near-continuous before/after measurement of air quality and health risk scores following a planned intervention (e.g., the instructions provided for responding to the health risk, as described herein), with statistical evaluation of whether the desired effect was achieved. Where an intervention falls short of targets (e.g., based on feedback information as described herein), the predictive analysis platform 1102 may generate an updated predictive analysis to support plan adjustment and iterative improvement via an adaptive intervention feedback loop (e.g., a loop comprising some or all of steps 1202-1214, as described herein).

In some instances, the predictive analysis may include audience-adaptive narratives comprising structured air quality and health impact summaries calibrated to the knowledge level and priorities of the intended reader. The audience-adaptive narrative generation may span members of the public, medical and public health professionals, air quality scientists, urban and traffic planners, insurers, pharmaceutical researchers and/or other audiences for predictive health analyses as described herein. The predictive analysis may include a structured insight schema with a consistent output format (e.g., summary, key findings, health implications, recommendations) enabling integration into downstream reporting, clinical, or policy workflows. In some instances, the predictive analysis may include near-term (e.g., on the order of hours, days, or weeks as opposed to months or years) air quality information comprising near-term air quality outlooks. The near-term air quality information may be communicated through multiple mediums of the predictive analysis, including plain-language forecasts, graphs, alerts, charts, visual representations, haptic feedback, music, storytelling, art, and/or other mediums, when the predictive analysis it output as described herein.

The predictive analysis may additionally or alternatively include a summary of the health risk (e.g., a heightened risk of a disease in a county, city, and/or other geographic region). In some instances, the predictive analysis may track the flow and dissipation of pollution, including how long it takes to dissipate harmful chemicals, gases, or the like, and may include a flow map of this dissipation. The predictive analysis may additionally or alternatively include one or more instructions for responding to the health risk. The one or more instructions for responding to the health risk may include one or more of: a visual representation of geographic areas associated with the health risk (which may, e.g., comprise the mapping generated by the predictive analysis platform 1102), a suggested route for avoiding the health risk, a recommendation for placement of environmental sensors for gathering information of the health risk, an agenda, schedule, or report on event conditions, a text description of the health risk, or an audio description of the health risk. The predictive analysis may additionally or alternatively include the personal sensitivity profile identifying the specific pollutants, concentrations, and durations that trigger a measurable response in that individual, which may differ significantly from population averages as described herein. The predictive analysis may additionally or alternatively include the per-building infiltration coefficient that updates dynamically and the predictive indoor exposure alert as described herein. The predictive analysis may additionally or alternatively include the indoor source and behavior fingerprinting as described herein. The predictive analysis may additionally or alternatively include groupings and correlated behaviors across distributed sensor networks as described herein.

At step 1214, the predictive analysis platform 1102 may output, via the communication interface to a computing device, the predictive analysis. In outputting the predictive analysis, the predictive analysis platform 1102 may send one or more commands to the computing device, wherein the one or more commands cause the computing device (e.g., the user device 1108) to display a user interface comprising the predictive analysis. The computing device may be the user device 1108 and/or a wearable device as described herein. The computing device may be managed by a pharmaceutical organization, a healthcare organization (e.g., a hospital or pathology lab), and/or any other organization or enterprise providing healthcare services or disaster prevention services. In some instances, the predictive analysis may include population-level comparative scoring, where individual risk scores are benchmarked against population cohorts, enabling risk stratification relevant to insurance underwriting, pharmaceutical research, and clinical care teams. The predictive analysis may additionally include person-to-person comparison of health risks and analyses. In these instances, the predictive analysis platform 1102 may output the predictive analysis to a device of a person associated with the user or wearer of the wearable device (e.g., a relative, a co-patient, a caregiver, and/or other associated persons). By outputting the predictive analysis to a device (e.g., wearable device, mobile phone, laptop, or the like) of a person associated with the user or wearer of the wearable device that provided at least a portion of the measurement information at step 1202, the predictive analysis platform 1102 may facilitate optimized individual behavior and promote healthy living by informing individuals associated with the wearer of the wearable device of health risks corresponding to the wearer of the wearable device. The predictive analysis platform 1102 may receive, from the computing device based on the displaying the user interface, feedback information corresponding to the predictive analysis, wherein the feedback information is used to update the predictive analysis model. For example, a user of the computing device may provide feedback indicating the accuracy of the predictive analysis. If a predictive analysis indicating a health risk is confirmed to be accurate, the predictive analysis model may be updated by the predictive analysis platform 1102 causing the predictive analysis model to store one or more correlations between the set of measurement information used to generate the risk scores, so that measurement information received by the predictive analysis platform 1102 will be given the same weight in the future and provide a similar contribution to the generation of the risk score. If a predictive analysis indicating a health risk is determined to be inaccurate, the predictive analysis platform 1102 may update the predictive analysis model by providing new training data or prompts to cause the predictive analysis model to modify its calculation of the risk scores in future predictive analyses.

Additionally or alternatively, the predictive analysis platform 1102 may output an alert corresponding to the health risk and the predictive analysis. The alert may include one or more of: a haptic alert for a wearable device, an audio alarm (e.g., a tone, music, storytelling, speech, or the like), a visual alert (e.g., a text message, art, graphic design, or the like) or a phone call. In some instances, a wearable device may be configured to produce a haptic response (e.g., a vibration, buzz, pulse, and/or other haptic responses) based on receiving the alert. The alert may additionally or alternatively be synchronized to a calendar on the wearable device. Providing the predictive analysis to the computing device may equip consumers, health insurers, pharmaceutical companies, and/or other entities to understand the risk of where individuals live, who they cover, and what medicines may have the greatest beneficial impact.

Based on outputting the predictive analysis, the predictive analysis platform 1102 may update the predictive analysis model. In updating the predictive analysis model, the predictive analysis platform 1102 may refine and/or otherwise update the predictive analysis model to improve the accuracy in generating risk scores and predictive analyses based on measurement information. For example, the predictive analysis platform 1102 may update the predictive analysis model by storing correlations or providing new training data or prompts as described herein. The predictive analysis model may evolve over time to learn based on changes in region, inputs, and level of data collection, improving upon the model over time. In some instances, wearable sensors may have a direct API connection to a cloud environment for more seamless data evaluation, allowing the predictive analysis model to be continuously or near-continuously updated in real time with new inputs from the wearable sensors.

One or more aspects of the disclosure may be embodied in computer-usable data or computer-executable instructions, such as in one or more program modules, executed by one or more computers or other devices to perform the operations described herein. Generally, program modules include routines, programs, objects, components, data structures, and the like that perform particular tasks or implement particular abstract data types when executed by one or more processors in a computer or other data processing device. The computer-executable instructions may be stored as computer-readable instructions on a computer-readable medium such as a hard disk, optical disk, removable storage media, solid-state memory, RAM, and the like. The functionality of the program modules may be combined or distributed as desired in various embodiments. In addition, the functionality may be embodied in whole or in part in firmware or hardware equivalents, such as integrated circuits, application-specific integrated circuits (ASICs), field programmable gate arrays (FPGA), and the like. Particular data structures may be used to more effectively implement one or more aspects of the disclosure, and such data structures are contemplated to be within the scope of computer executable instructions and computer-usable data described herein.

As described herein, the various methods and acts may be operative across one or more computing servers and one or more networks. The functionality may be distributed in any manner, or may be located in a single computing device (e.g., a server, a client computer, and the like). For example, in alternative embodiments, one or more of the computing platforms discussed above may be combined into a single computing platform, and the various functions of each computing platform may be performed by the single computing platform. In such arrangements, any and/or all of the above-discussed communications between computing platforms may correspond to data being accessed, moved, modified, updated, and/or otherwise used by the single computing platform. Additionally or alternatively, one or more of the computing platforms discussed above may be implemented in one or more virtual machines that are provided by one or more physical computing devices. In such arrangements, the various functions of each computing platform may be performed by the one or more virtual machines, and any and/or all of the above-discussed communications between computing platforms may correspond to data being accessed, moved, modified, updated, and/or otherwise used by the one or more virtual machines.

Aspects of the disclosure have been described in terms of illustrative embodiments thereof. Numerous other embodiments, modifications, and variations within the scope and spirit of the appended claims will occur to persons of ordinary skill in the art from a review of this disclosure. For example, one or more of the steps depicted in the illustrative figures may be performed in other than the recited order, and one or more depicted steps may be optional in accordance with aspects of the disclosure.

What is claimed is:

1. A computing platform comprising:
at least one processor;
a communication interface communicatively coupled to the at least one processor; and
memory storying computer-readable instructions that, when executed by the at least one processor, cause the computing platform to:
receive, via the communication interface from one or more measurement information sources, a set of measurement information corresponding to health parameters at a geographic location;
generate, using a predictive analysis model and the set of measurement information, one or more risk scores indicating a likelihood of a presence of a health risk at the geographic location;
generate a mapping comprising the one or more risk scores and the geographic location;
generate, using the predictive analysis model and based on the mapping and the one or more risk scores, a predictive analysis comprising:
an explanation of the one or more risk scores; and
an indication of the health risk at the geographic location; and
output, via the communication interface to a computing device, the predictive analysis.

2. The computing platform of claim 1, wherein the one or more measurement information sources comprise one or more wearable devices comprising integrated sensors for detecting:
anonymized biometric information of a wearer of the wearable device;
one or more measured variables corresponding to one or more attributes of a known particle; and
one or more additional environment variables.

3. The computing platform of claim 1, wherein the instructions, when executed by the at least one processor, further cause the computing platform to:
train, based on historical measurement information, the predictive analysis model, wherein the training of the predictive analysis model configures the predictive analysis model to:
generate the one or more risk scores by comparing the set of measurement information to portions of the historical measurement information indicating a given health risk;
generate the mapping; and
generate, based on the mapping and the one or more risk scores, the predictive analysis; and
update, based on the outputting the predictive analysis, the predictive analysis model.

4. The computing platform of claim 1, wherein:
the predictive analysis model comprises a generative artificial intelligence model, and
the instructions, when executed by the at least one processor, configure the computing platform to generate the one or more risk scores by providing a prompt to the predictive analysis model, the prompt comprising:
the set of measurement information; and
one or more instructions for the predictive analysis model to generate the one or more risk scores based on historical information indicating a given health risk.

5. The computing platform of claim 1, wherein the set of measurement information comprises:
information gathered by a wearable device, wherein the information gathered by the wearable device comprises one or more of:
particulate matter measurements, chemical measurements, heavy metal measurements, gas measurements, environmental toxin measurements, per- and polyfluoroalkyl substance (PFAS) measurements, biometric information of a wearer of the wearable device, audio measurements, wind speed, wind direction, particle concentration, gas concentration, temperature, relative humidity, air pressure, dew point, vibration frequency, noise level, ventilation status, altitude, topographical information, occupancy, traffic counts, mold growth information, mildew growth information, population information, luminosity information, precipitation information, bacteria detection information, or virus detection information;
information gathered by one or more environmental sensors, wherein the information gathered by the one or more environmental sensors comprises one or more of:
particulate matter measurements, chemical measurements, heavy metal measurements, gas measurements, environmental toxin measurements, per- and polyfluoroalkyl substance (PFAS) measurements, audio measurements, wind speed, wind direction, particle concentration, gas concentration, temperature, relative humidity, air pressure, dew point, vibration frequency, noise level, ventilation status, altitude, topographical information, occupancy, traffic counts, mold growth information, mildew growth information, population information, luminosity information, precipitation information, bacteria detection information, or virus detection information; and
information corresponding to a healthcare service provider, wherein the information corresponding to the healthcare service provider comprises one or more of:
therapeutic information for treating diseases, pathology information, community disease information, infection rates for a disease or virus, or infection vectors for a disease or virus.

6. The computing platform of claim 1, wherein the one or more measurement information sources comprise one or more of:
a particle sensor, a network of sensors located at different geographical locations, a repository of information gathered by multiple unassociated sensors, satellites gathering environmental information, a repository of automotive traffic information, one or more interior devices gathering environment information of an interior of a building, one or more vehicle devices gathering environment information from a moving vehicle, one or more anemometers, or a database corresponding to an enterprise organization.

7. The computing platform of claim 1, wherein the instructions, when executed by the at least one processor, cause the computing platform to generate the one or more risk scores by:
providing the set of measurement information to the predictive analysis model to cause the predictive analysis model to determine, based on applying weighted values to portions of the set of measurement information, the likelihood of the presence of the health risk; and
generate, by converting the likelihood to a numerical value, a risk score, of the one or more risk scores, for at least one of:
an individual;
a population; and
an environment corresponding to the geographic location.

8. The computing platform of claim 1, wherein the instructions, when executed by the at least one processor, cause the computing platform to generate the predictive analysis by providing one or more instructions to the predictive analysis model to cause the predictive analysis model to generate the explanation of the one or more risk scores, wherein the explanation comprises information indicating portions of the set of measurement information that contributed to each of the one or more risk scores.

9. The computing platform of claim 1, wherein the instructions, when executed by the at least one processor, cause the computing platform to output the predictive analysis by:
sending one or more commands to the computing device, wherein the one or more commands cause the computing device to display a user interface comprising the predictive analysis; and
receiving, from the computing device based on the displaying the user interface, feedback information corresponding to the predictive analysis, wherein the feedback information is used to update the predictive analysis model.

10. The computing platform of claim 1, wherein the predictive analysis further comprises one or more instructions for responding to the health risk, the one or more instructions for responding to the health risk comprising:
a visual representation of geographic areas associated with the health risk;
a suggested route for avoiding the health risk;
a recommendation for placement of environmental sensors for gathering information of the health risk;
a text description of the health risk; or
an audio description of the health risk.

11. The computing platform of claim 1, wherein the instructions, when executed by the at least one processor, cause the computing platform to output the predictive analysis by:
outputting an alert corresponding to the health risk and the predictive analysis, wherein the alert comprises one or more of:
a haptic alert for a wearable device;
an audio alarm;
electronic mail;
a text message; or
a phone call.

12. A method comprising, by a computing platform comprising at least one processor, a communication interface, and memory:
receiving, via the communication interface from one or more measurement information sources, a set of measurement information corresponding to health parameters at a geographic location;
generating, using a predictive analysis model and the set of measurement information, one or more risk scores indicating a likelihood of a presence of a health risk at the geographic location;
generating a mapping comprising the one or more risk scores and the geographic location;
generating, using the predictive analysis model and based on the mapping and the one or more risk scores, a predictive analysis comprising:
an explanation of the one or more risk scores; and
an indication of the health risk at the geographic location; and
outputting, via the communication interface to a computing device, the predictive analysis.

13. The method of claim 12, wherein the one or more measurement information sources comprise one or more wearable devices comprising integrated sensors for detecting:
anonymized biometric information of a wearer of the wearable device;
one or more measured variables corresponding to one or more attributes of a known particle; and
one or more additional environment variables.

14. The method of claim 12, further comprising:
training, based on historical measurement information, the predictive analysis model, wherein the training of the predictive analysis model configures the predictive analysis model to:
generate the one or more risk scores by comparing the set of measurement information to portions of the historical measurement information indicating a given health risk;
generate the mapping; and
generate, based on the mapping and the one or more risk scores, the predictive analysis; and
updating, based on the outputting the predictive analysis, the predictive analysis model.

15. The method of claim 12, wherein:
the predictive analysis model comprises a generative artificial intelligence model, and
wherein the generating the one or more risk scores comprises providing a prompt to the predictive analysis model, the prompt comprising:
the set of measurement information; and
one or more instructions for the predictive analysis model to generate the one or more risk scores based on historical information indicating a given health risk.

16. The method of claim 12, wherein the generating the one or more risk scores comprises:
providing the set of measurement information to the predictive analysis model to cause the predictive analysis model to determine, based on applying weighted values to portions of the set of measurement information, the likelihood of the presence of the health risk; and
generating, by converting the likelihood to a numerical value, a risk score, of the one or more risk scores, for at least one of:
an individual;
a population; and
an environment corresponding to the geographic location.

17. The method of claim 12, wherein the generating outputting the predictive analysis comprises:
sending one or more commands to the computing device, wherein the one or more commands cause the computing device to display a user interface comprising the predictive analysis; and
receiving, from the computing device based on the displaying the user interface, feedback information corresponding to the predictive analysis, wherein the feedback information is used to update the predictive analysis model.

18. The method of claim 12, wherein the predictive analysis further comprises one or more instructions for responding to the health risk, the one or more instructions for responding to the health risk comprising:
a visual representation of geographic areas associated with the health risk;
a suggested route for avoiding the health risk;
a recommendation for placement of environmental sensors for gathering information of the health risk;
a text description of the health risk; or
an audio description of the health risk.

19. The method of claim 12, wherein the outputting the predictive analysis comprises:
outputting an alert corresponding to the health risk and the predictive analysis, wherein the alert comprises one or more of:
a haptic alert for a wearable device;
an audio alarm;
electronic mail;
a text message; or
a phone call.

20. A system comprising a plurality of sensors and a computing platform:
wherein at least one first sensor of the plurality of sensors is integrated into a wearable device and is configured to transmit a first portion of measurement information to the computing platform;
wherein at least one second sensor of the plurality of sensors comprises a transmitter and a detector, is located in a respective one of a plurality of geographic locations, and is configured to:
determine, using the detector, a second portion of measurement information corresponding to health parameters in the respective one of the plurality of geographic locations of the at least one second sensor, and
transmit the second portion of measurement information corresponding to health parameters to the computing platform; and
wherein the computing platform comprises a communication interface, a processor, and a memory storing computer-readable instructions that, when executed by the processor, causes the computing platform to:
receive, via the communication interface from the at least one first sensor and the at least one second sensor, a set of measurement information comprising the first portion and the second portion;
generate, using a predictive analysis model and the set of measurement information, one or more risk scores indicating a likelihood of a presence of a health risk at the geographic location;
generate a mapping comprising the one or more risk scores and the geographic location;
generate, using the predictive analysis model and based on the mapping and the one or more risk scores, a predictive analysis comprising:
an explanation of the one or more risk scores; and
an indication of the health risk at the geographic location; and
output, via the communication interface to a computing device, the predictive analysis.
